(19) United States
(12) Patent Application Publication
(10) Pub. No.: US 2004/0009947 A1
(43) Pub. Date: Jan. 15, 2004

(54) Compounds and methods for enhancing delivery of free polynucleotide

(76) Inventors: Robert W. Malone, Rockville, MD (US); Jill G. Malone, Rockville, MD (US)

Correspondence Address: FOLEY AND LARDNER, SUITE 500, 3000 K STREET NW, WASHINGTON, DC, 20007, US

(21) Appl. No.: 10/445,034

(22) Filed: May 27, 2003

Related U.S. Application Data
(63) Continuation of application No. 09/763,088, filed on May 3, 2001, now abandoned.
Application No. 09/763,088 is a 371 of international application No. PCT/US99/18726, filed on Aug. 19, 1999.
(60) Provisional application No. 60/097,098, filed on Aug. 19, 1998.

(51) Int. Cl. A61K 48/00; C12P 21/02; C12N 15/88; C12N 15/85; C12N 5/08

(52) U.S. Cl. 514/44; 435/455; 435/366; 435/458

(57) ABSTRACT

The discovery of simple, nontoxic, and pharmaceutically defined methods for genetic modification of cells and tissues would enable development of a variety of molecular medicines. “Free”, ‘direct’, or ‘naked’ polynucleotide administration is a simple, apparently safe, and pharmaceutically defined polynucleotide delivery method. Murine, macaque, and clinical human experiments have demonstrated transfection of various tissues, such as respiratory tissues, after direct application of ‘free’ polynucleotide. However, direct DNA transfection is relatively inefficient in comparison to many transduction systems. The invention herein is directed to transfection enhancing agents which augment the transfection activity of ‘free’ polynucleotide, thereby facilitating the development of simple and safe alternatives to tissue transfection, more particularly respiratory tissue transfection. The experiments described herein indicate that nucleases, both extra- and intra-cellular, present in many biological fluids, such as respiratory fluid, accelerate clearance of biologically active plasmid from the tissue, and that co-administration of a nuclease inhibitor together with free polynucleotide results in marked enhancement of expression of the polynucleotide of interest. These findings support the disclosed invention of an improved polynucleotide delivery system, a ‘free’ plasmid-based transfection technology.

### CROSS-REFERENCE TO RELATED APPLICATIONS

This application claims priority to co-pending U.S. Provisional Application Serial No. 60/097,098, filed Aug. 19, 1998. In addition, this application relates to co-pending U.S. patent application Ser. No. 08/862,632, filed May 23, 1997, entitled ‘DNA Vaccines For Eliciting A Mucosal Immune Response’. The disclosures of both these applications are hereby incorporated by reference in their entirety.

### RESEARCH SUPPORT

The development of the present invention was supported by the University of Maryland, Baltimore and the University of Maryland Medical System. Certain experiments described herein were supported by NIH grants R01 RR12307 (RWM) and K02 AI01370 (RWM). The Federal Government may have certain rights in the invention.

### TECHNICAL FIELD AND INDUSTRIAL APPLICATIONS

The field of the invention generally relates to the use of transfection enhancing agents to enhance the polynucleotide transfer and expression associated with direct delivery of ‘free’ polynucleotide preparations to target cells or tissues. The field of the invention more specifically relates to nuclease inhibitors which prevent the extracellular and intracellular cleavage and degradation of the free polynucleotide. In a more specific embodiment, the invention relates to the use of direct competitive nuclease inhibitors, such as aurin tricarboxylic acid (ATA), a nuclease and apoptosis inhibitor, to enhance respiratory, mucosal, and skin tissue transfection. By augmenting the transfection efficiency of free polynucleotide, the invention allows for simple and safe alternatives to biological vector-based delivery and transfection techniques. The invention allows for both in vitro and in vivo transfection. Utilities include but are not limited to the engineering of cultured prokaryotic and eukaryotic cells, bioreactor protein production, plynucleotide therapies for use in conjunction with organ transplantation, angioplasty, skin transplantation, and other clinical treatments which will benefit from genetic therapies.

Both the in vitro and in vivo application of the invention finds utility for use in pharmaceutical discovery, testing, target validation, and other drug development processes.

### BACKGROUND OF THE INVENTION

The delivery of endogenous and foreign genes to animal tissue for gene therapy has shown significant promise in experimental animals and volunteers. In particular, polynucleotide vaccines have been used to successfully immunize against influenza both in chickens and ferrets and against Plasmodium yoelii, rabies, human carcinoembryonic antigen and hepatitis B in mice. The commercial application of gene delivery technology to animal cells is broad and includes delivery of vaccine antigens, immunotherapeutic agents, and gene therapeutic agents [See generally Thomas & Capecchi, Cell, 51:503-512 (1987); Bertling, Bioscience Reports, 7:107-112 (1987); Smithies et al., Nature, 317:230-234 (1985)].

Although the utility of tissue transfection for prophylactic and therapeutic purposes has been scientifically confirmed, simple, efficient, and safe methods for in vivo polynucleotide delivery have yet to be fully developed. To date, polynucleotide delivery systems fall into two categories: biological and synthetic vectors. Biological vector systems utilize a biological organism, such as a virus or a bacteria, to effect the delivery and transfer of a polynucleotide of interest to target tissues within a host organism. Synthetic vector systems rely on endogenous cellular pathways (such as endocytosis) to effect delivery and transfer of the polynucleotide of interest. The synthetic vector preparation may be as simple as a plasmid or further include a polynucleotide complexed to colloidal materials (such as cationic lipids and liposomes).

There are advantages and drawbacks associated with each delivery system. For example, from the perspective of efficiency of delivery and expression, biological vector preparations are superior. Biological vector systems take advantage of a biological mechanism that facilitates integration of the polynucleotide of interest into the host genome, thereby allowing the polynucleotide of interest to be repeatedly and continuously expressed not only in the initial cell transfected but in subsequent daughter cells. Alternatively, absent inclusion of additional viral components to the vector, synthetic vector systems are generally incapable of providing for stable integration of the polynucleotide of interest. Therefore, repeat administration is often required to achieve the desired systemic response. Furthermore, whereas biological vector preparations frequently utilize a biological mechanism inherent in the vector to target specific tissues, synthetic vector preparations generally lack such a mechanism and must be directly delivered to target tissue. However, it is important to note that biological vectors (and synthetic vectors incorporating viral components) are only efficient when the tissue in question expresses the appropriate receptor for the virus or bacteria. Biological vectors frequently fail in vivo due to the lack of expression of the appropriate receptor.

The picture is reversed when one evaluates the two systems from the perspective of safety and simplicity. Biological vector polynucleotide transfer systems, particularly adenovirus and adeno associated virus (AAV) vectors, are most widely used for pre-clinical studies. However, large scale GMP manufacturing and distribution, and general safety issues have confined their clinical use to a relatively small number of trials involving life threatening disease which is refractory to established treatments. Synthetic vector polynucleotide transfer systems, particularly liposomal preparations, are safer and more amenable to large scale manufacture and distribution. Whereas most cell biology laboratories routinely prepare two component lipoplex formulations for transfection of cultured cells, sophisticated technology is required for generating recombinant adenovirus or AAV vectors as well as for propagating large scale, high titer, helper-free stocks of recombinant vectors.

However, not all synthetic vector systems are equivalent. Those that utilize liposomal packaging and the like frequently suffer from unacceptably low transfection efficiency. In fact, most are not effective in vivo. The simplest and one of the safest in vivo polynucleotide delivery systems involves the direct application of high concentration ‘free’ or ‘naked’ polynucleotides (typically mRNA or DNA). The simplicity and reproducibility of direct in vivo polynucleotide transfer has led those skilled in the art to adopt the technique, particularly for stimulating immune responses to plasmid-encoded proteins [see www.genweb.com/Dnavax/dnavax.html (Whalen, 1998) for a comprehensive reference list].

Biologic clearance of polynucleotide delivery preparations in vitro and in vivo is a critical determinant of transfection or transduction efficiency and one that affects all polynucleotide delivery systems. The biological clearance may occur before the polynucleotide preparation has reached the target tissue or cell, via extracellular nucleases. Alternatively, the clearance may occur after the polynucleotide preparation has entered the cell but before it has entered the nucleus or acted on the intracellular target, via intracellular nucleases. While lipidic delivery systems and viral packaging provide protection from endolytic degradataion by endogenous nucleases, ‘free’ polynucleotides are susceptible to inactivation via endo- or exonucleolytic cleavage (by both extra- and intracellular nucleases). In those instances in which the treatment induces cell damage (such as needle injection of significant volumes), release of nucleases from damaged cells can compound this effect. However, viral vector systems are often subject to rapid nonspecific (e.g., complement) and specific (e.g., antibody neutralization, CTL) immunologic clearance. Likewise, lipoplexes are efficiently cleared by the reticuloendothelial system, and may also be subject to complement-mediated effects. Assuming that the biological pathway involved in transfection or transduction is not regulated, reduction of biological clearance of a polynucleotide delivery formulation both in vitro and in vivo should correlate with greater polynucleotide transfer efficiency by exposing tissue to a higher concentration of vector, lipoplex, or free polynucleotide for a longer period of time.

The invention herein overcomes the problem of biologic clearance of free polynucleotide preparations, thereby enhancing the delivery and expression of the polynucleotide of interest. Specifically, the invention involves the use of nuclease inhibitors to prevent the nucleolytic degradation of free polynucleotides. The invention finds utility for both in vitro and in vivo transfection. A number of pharmaceutical agents which indirectly inhibit nuclease activity by reducing the effective concentration of enzymatically required divalent cations are known in the art. Examples include ethylenediaminetetraacetic acid (EDTA) and citrate. However, the invention herein specifically utilizes agents which directly inhibit extracellular and intracelluar nucleases. Such agents include various competitive inhibitors including nucleotide analogs and aurin tricarboxylic acid (ATA).

### SUMMARY OF THE NVETION

It is a object of the invention to provide a method for enhancing the direct delivery of polynucleotides. The method utilizes the inhibition of extracellular and intracellular nuclease activity. This method finds utility both in vitro and in vivo transfection.

In a particular embodiment, the invention relates to transfection enhancing agents that allow for improved transfer of free polynucleotide preparations to targeted tissues of a host organism, thereby increasing the level of expression of the polynucleotide of interest in situ.

The method involves the administration of transfection enhancing agents in the form of direct competitive nuclease inhibitors in combination with free polynucleotide preparations; the combination allowing for more efficient transfection of a polynucleotide of interest.

In a preferred embodiment, the competitive nuclease inhibitor is aurin tricarboxylic acid or a functional derivative thereof.

In one embodiment, the polynucleotide preparations of the present invention may be used to enhance the transfer of free polynucleotide preparations to eukaryotic or prokaryotic cells in culture, thereby enhancing the level of expression of the polynucleotide (or polynucleotides) of interest.

In an alternate embodiment, the polynucleotide preparations of the present invention may be used for gene therapy in general, more specifically for delivering exogenous copies of a therapeutic gene or polynucleotide to a specific cellular or tissue target in vivo. Enhanced polynucleotide delivery also finds utility not only in vaccine therapies, such as polynucleotide vaccines, mucosal and intradermal polynucleotide vaccines, but also genetic therapies for inborn metabolic diseases, such as cystic fibrosis, and expression of immunomodulatory agents, such as cytokines or costimulatory molecules, and delivery of such therapeutic polynucleotides into cells of the immune system including antigen presenting cells.

The examples described in detail herein confirm that a diverse range of species including murine, rat and macaque respiratory tissues may be transfected by simple direct application of plasmid. The examples further confirm that rodent, primate and human tissues express DNAse activity, that this activity may be inhibited, and that inhibition of such activity enhances the transfection of respiratory tissues. Central to these results is the inventors' discovery that ATA co-administration enhances this transfection activity. Thus, the simple yet robust polynucleotide delivery technique embodied by the present invention opens new developmental avenues for gene therapy applications, particularly genetic modification of respiratory tissues.

### BRIEF DESCRIPTION OF THE DRAWINGS

FIG. 1: Depicts assays preformed using lavage fluid obtained from 4 different mice and demonstrates the presence of significant levels of nuclease activity in the murine lung lavage fluid.

FIGS. 2A-2B: Depicts the degradation of intratracheally administered plasmid, demonstrating the effect of ATA on this process in vitro. FIG. 2A depicts the results associated with lavage fluid. FIG. 2B depicts the results associated with tissue extract. See Table 1 for the details of the protocol.

FIGS. 3A-3B: Depicts the experimental results of the Balb-C mouse lung transfection resulting from co-administration of ATA and pND2Lux plasmid. FIG. 3A depicts the ratio ATA to free DNA at each dosage, from 0.1 [g/g to 6 [g/g. FIG. 3A also shows the negative results associated with doses of EDTA and sodium citrate. FIG. 3B depicts the same experimental results from FIG. 3A associated with doses of ATA, further including error bars.

FIG. 4: Depicts the optimization of efficacy of ATA/plasmid administration, showing the mean and standard deviation of total relative units (less background) obtained from groups of three similarly aged animals treated under the same conditions with same DNA at the same time. Each mouse was treated with 100 micrograms of pND2Lux in 100 microliters of water via intratracheal installation using the methods described in detail below.

FIG. 5: Depicts the transfection enhancing activity associated with c-administration of free plasmid with DNAse inhibitors ATA, EDTA, and citrate. ATA co-administration markedly augmented the levels of reporter protein expression detected after intratracheal administration of plasmid. Neither EDTA nor citrate significantly enhanced the level of expression. In fact, administration with EDTA was associated with significant toxicity.

FIG. 6: Depicts a map of the expression vector (pND) constructed to contain the following elements: (1) the CMV immediate early promoter (HCMV IE1), (2) the CMV IE1 intron, (3) a cloning site, and (4) the RNA terminator/polyadenylation site from bovine growth hormone (BGH). These elements are contained in a pUC19 replicon.

FIG. 7: Depicts the results, in terms of luciferase expression, of intradermal injection of luciferase DNA into rat skin, both with and without ATA.

FIG. 8: Depicts the results, in terms of luciferase expression, of intradermal transfection of luciferase DNA into mouse skin, both with and without ATA.

FIG. 9: Depicts the histology of treated mouse lung tissue obtained from control and treated mice. Images include representative low (4×) and intermediate (20×) and high (40×) power fields of hematoxilyn and eosin stained lung sections. Tissues were fixed, embedded, sectioned, and stained two days after treatment yet prior to staining. Note the absence of polymorphonuclear cells indicating a lack of acute inflammatory response, the presence of intact respiratory epithelium lining conducting airway, and the lack of significant fluid or inflammatory cell infiltrates within the parenchyma.

FIGS. 10A-10C: Depicts the levels of DNAse activity observed in murine (10A), macaque (10B), and human (10C) lung fluids and that ATA inhibits lung DNAse activity in vitro.

### DETAILED DESCRIPTION OF THE INVENTION

I. Polynucleotide Delivery

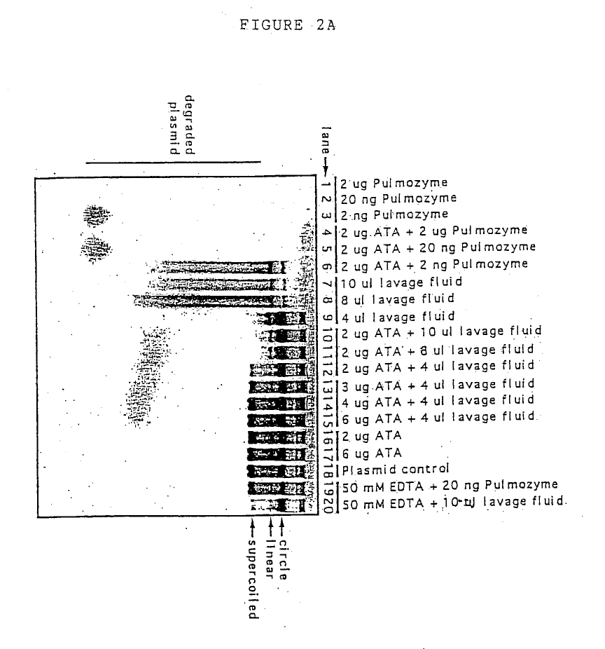

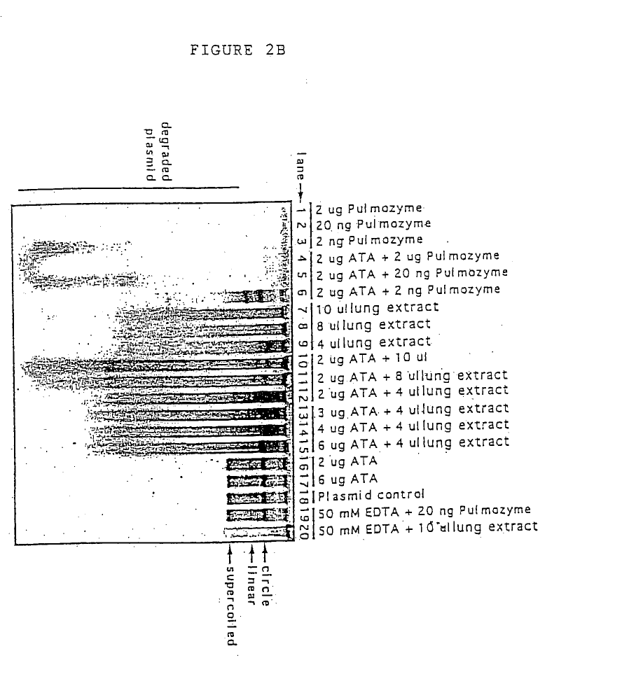

The rate of clearance is frequently an important determinant of in vivo drug activity. As discussed above, multiple mechanisms may contribute to clearance of extracellular polynucleotides. In the case of respiratory mucosa, these include nuclease-mediated degradation, mucous entrapment with mucociliary clearance, phagocytic clearance, and absorption and distribution to non-respiratory tissues. In the examples described herein, the presence of nuclease activity in tissues and fluids was demonstrated in vitro by incubating plasmid with lung extracts and lavage fluids and in vivo in various animal systems by direct intradermal, intramuscular and intratracheal plasmid administration and electrophoretic analysis of lavage or tissue recovered. In addition to extracellular nucleases, it is also documented in the literature and documented in FIG. 2A that intracellular (predominantly cytoplasmic) nucleases also contribute to intracellular clearance of transfected polynucleotides. The focus of the instant invention is that addition of nuclease inhibitors to applied plasmid significantly improves transfection by reducing nuclease-mediated clearance.

Murine, rat and primate models were used for the proof of principle experiments described herein. It was discovered that co-administration of plasmid with the nuclease inhibitor ATA markedly boosted subsequent reporter protein expression relative to free DNA treatment. These observations further demonstrate that the transfection activity associated with direct application of nucleic acids to tissues can be significantly enhanced by agents which reduce extracellular or intracellular clearance of exogenously introduced polynucleotide plasmid by reducing nuclease-mediated clearance.

Low levels of transfection associated with the treatment of rodent, nonhuman primate, and human respiratory tissues with ‘free’ plasmid is widely discussed in the art. This inefficiency has limited the utility of direct DNA and RNA delivery. Clinical trials involving direct polynucleotide delivery have not progressed beyond phase 2 due to this lack of efficacy. It is important to note that no significant toxicity has been observed in these trials.

Multiple viral and non-viral polynucleotide delivery systems are being developed and adapted for various clinical gene therapy applications. Each of these delivery systems and therapeutic implementations may be considered as pharmaceuticals, and can be described in terms of relative efficacy and toxicity.

Efficacy may be defined in terms of reporter protein expression, or may be defined in the context of clinical response (which in turn reflects the level of protein expression necessary to generate a response). Toxicity includes both short term deleterious effects (atelectasis, enhanced vascular permeability, or non-therapeutic inflammation, apoptosis, necrosis), as well as long term risks (fibrosis, insertional mutagenesis/carcinogenesis, cellular cytotoxicity and/or autoimmunity). The clinical utility of any polynucleotide delivery system will be a function of the intended application and the ratio of efficacy and toxicity of the system.

Technologies which provide efficient, nontoxic methods for genetic modification of tissues will enable development of a variety of genetic medicines. Such medicines may provide treatments for inborn errors of metabolism such as cystic fibrosis, acute treatments for various pulmonary disorders such as introgenic pulmonary fibrosis, and prophylactic treatments such as mucosal and parenteral polynucleotide vaccination. In another general application, simple and safe gene delivery methods may be particularly useful for analysis of gene function and for validation of potential pharmaceutical targets. Both pre-clinical (mouse, rat, macaque) experiments and human clinical trials have demonstrated that tissues may be genetically modified by administration of ‘free’ or ‘naked’ polynucleotides [Balasubramaniam et al., Gene Therapy; 3(2):163-172 (1996) Malone et al., Advances, Challenges And Applications for Self-Assembling Systems, 4.1.1 (1996); Meyer et al., Gene Ther, 2(7):450-460 (1995); Tsan et al., Am J Physiol, 268 (6Pt 1): 1052-L1056 (1995); Zabner et al., Clin Invest, 100(6):1529-1537 (1997)]. Although this process is inefficient and poorly characterized, it appears to have little short term toxicity. If efficiency were improved without additional toxicity, ‘free’ DNA administration to tissues might become clinically effective if adapted for some of the above applications. The data presented herein supports development of ‘free’ DNA transfection enhancing agents which inhibit or delay clearance of administered plasmid as one strategy for improving the efficiency of this simple transfection method.

II. Elements of the Invention

A. Transfection Enhancing Agents

The invention employs the co-administration of transfection enhancing agents with free polynucleotide preparations, the enhancing agent acting as a protective agent, preventing the nucleolytic degradation of the polynucleotide by the endogenous nucleases. The transfection enhancing agents contemplated by the instant invention are nuclease inhibitors, preferably direct competitive nuclease inhibitors. Although the agent need not be a specific nuclease inhibitor, it is preferable that the agent have activity against those nucleases found in the target cells and tissues of the host organism.

Nucleases are enzymes which break the linkages (phosphodiesters) between nucleic acids in a polynucleotide chain. Nucleases are found both intracellular (e.g. cytoplasmic nucleases) and extracellular (e.g. circulatory or systemic nucleases). The instant invention is primarily concerned with nucleases that are endogenous to the target system, both intra- and extracellular. Nucleases may be specific to RNA or DNA, to internal or external sites. RNAses or ribonucleases are nucleases that catalyze the breaking of some linkages between nucleotide in RNA. DNAses or deoxyribonucleases are nucleases that hydrolyze the interior bonds of deoxyribonucleotides and strings them together into oligonucleotides or polynucleotides. For example, pancreatic DNAse I yields di- and oligo-nucleotide 5-phosphates while pancreatic DNAse II yields 3-phosphates. Restriction enzymes Or restriction ‘endonucleases’ are proteins that recognize specific, short polynucleotides and cuts DNA at those sites. Bacteria contain over 400 such enzymes that recognize and cut over 100 different DNA sequences.

Nucleases that cleave a polynucleotide substrate at the internal sites (the phosphodiester bonds) in the polynucleotide sequence are referred to as ‘endonucleases’. ‘Endoribonucleases’ are endonucleases that specifically hydrolyze the interior bonds of a ribonucleotide. An example of an endogenous endonuclease is endonuclease SI, a nuclease enzyme from the fungi Aspergillus oryzae which cuts the phosphodiester between nucleotides in single-stranded DNA or RNA, producing individual nucleotide molecules. Nucleases that cleave nucleotides sequentially from the free ends of a linear nucleic substrate are referred to as ‘exonucleases’. Examples of exonucleases include but are not limited to exonuclease III, an exonuclease which removes nucleotide one at a time from the 5′-end of duplex DNA which does not have a phosphorylated 3′-end; exonuclease VII, an exonuclease which makes oligonucleotide by cleaving chunks of nucleotide off of both ends of single-stranded DNA; and exonuclease lambda, an exonuclease that removes nucleotide from the 5′ end of duplex DNA which have 5′-phosphate groups attached to them.

Not all nuclease inhibitors are effective transfection enhancing agents for direct plasmid transfection of tissues, more particularly respiratory or mucosal tissues. The experiments described below include tests performed with direct and indirect nuclease inhibitors. Whereas ATA is a direct competitive nuclease inhibitor, both EDTA and citrate indirectly inhibit nucleases by sequestering the divalent cation cofactors required for nuclease activity. It was discovered that ATA is a potent direct transfection enhancing agent while EDTA and citrate did not augment free DNA transfection.

All three of these low molecular weight nuclease inhibiting pharmaceuticals are subject to rapid systemic absorption and distribution. While not wishing to be bound by theory, it is possible that differences in the concentration and distribution of target ligands can account for differences in in vivo transfection enhancing activity. In general, the absorption of proteins across respiratory epithelium is much less efficient than the diffusion of divalent cations. Therefore, the nuclease activity of respiratory fluids treated with ATA will be influenced by the ATA/nuclease binding constant and the concentration of ATA and nuclease within the respiratory space. In contrast, divalent cations are present at relatively high concentrations in both extracellular fluid and within cells. Unbound divalent cations rapidly equilibrate between all body compartments. Therefore, once a divalent cation nuclease cofactor within the lung is sequestered by EDTA or citrate, the complex may distribute systemically, and the depleted free cations will be rapidly replenished by equilibration with the large pool of unbound cation. In addition to these considerations, the polymerization of ATA to form a high molecular weight complex [Guo et al., Thromb Res 71(1):77-88 (1993)] may enhance retention within respiratory fluids, and this may also contribute to the enhanced activity of this drug. These processes of absorption, diffusion and equilibration may account for the observed in vivo differences between the activity of direct and indirect nuclease inhibitors.

Examples of naturally produced nuclease inhibitors include but are not limited to NuiA, a protein produced by Anabaena sp. [Muro-Pastor A M, J Mol Biol, 268(3):589-98 (May 9, 1997)] and DMI-2, an acid nuclease inhibitor and polyketide metabolite of Streptomyces sp. strain 560 [Ross G F, et al., Gene Ther, 5(9):1244-50 (September 1998)]. Examples of synthetic nuclease inhibitors include but are not limited to diethyl pyrocarbonate [Zsindely A, et al., Acta Biochim Biophys Acad Sci Hung, 5(4):423-34 (1970)].

In the context of the instant invention, the nuclease inhibitor is preferably a direct competitive nuclease inhibitor. Examples of competitive nuclease inhibitors include but are not limited to acridine dimers [Malvy C et al, Chem Biol Interact, 73(2-3):249-60 (1990)]; actin and actin derivatives [Macanovic M, et al., Clin Exp Immunol, 108(2):220-6 (May 1997)]; aurin tricarboxylic acid (abbreviated ATA or ACTA) [Hallick R B, et al, Nucleic Acids Res,4(9):3055-64 (1977)]; 5′-AMP, a competitive inhibitor of SI nuclease [Gite S, et al., Biochem J, 288:571-5 (Dec. 1, 1992)]. Additional endogenous nuclease inhibitors have been identified in and isolated from various biological tissues and/or fluids [Fominaya J M, et al., Biochem J, 253(2):517-22 (Jul. 15, 1988), RNAse inhibitor in rat testis; Lee F S, et al., Biochemistry, 28(1):225-30 (Jan. 10, 1989), RNAse inhibitor in human placenta; Gauthier D, et al., Neurochem Res, 12(4):335-9 (April 1987), RNAse inhibitors from yeast and liver; Turner P M, et al., Biochem Biophys Res Commnun, 114(3):1154-60 (Aug. 12, 1983), RNAse inhibitors from porcine thyroid and liver].

Examples of RNAse inhibitors include but are not limited to bromopyruvic acid, an inactivator of bovine pancreatic ribonuclease A [Wang MH, et al., Biochem J, 320 (Pt 1):187-92 (Nov. 15, 1996)]; retinoids, inhibitors of ribonuclease P activity [Papadimou E, et al., J Biol Chem, 273(38):24375-8 (Sep. 18, 1998)]; N-(4-tert-Butylbenzoyl)-2-hydroxy-1-naphthaldehyde hydrazone (BBNH), an inhibitor of RNAse H of HIV-1 RT [Borchow G, et al., Biochemistry, 36(11):3179-85 (Mar. 18, 1997)]; poly(2′-O-(2,4-dinitrophenyl)]poly(A)[DNP-poly(A)], an inhibitor of RNases A, B, S, T1, T2 and H [Rahman M H, et al., Anal Chem, 68(1):134-8 (Jan. 1, 1996)]; phosphorothioate oligonucleotides, inhibitors of RNase H [Gao W Y, et al., Mol Pharmacol, 41(2):223-9 (February 1992)] heparin, polyvinyl sulfate, and Diethylpyrocarbonate [Gauthier D, et al., (April 1987) supra].

Examples of DNAse inhibitors include but are not limited to 5838-DNI (or 1,4,4a,5,12,12 a-hexahydro-4,4a,11,12a-tetrahydroxy-3,8-dimethoxy-9-methoxycarbonyl-10-methyl-1,5,12-trioxo naphthacene), a competitive inhibitor of porcine spleen DNAse II produced by Streptomyces sp. Strain No. A-5838 and having a structure similar to tetracenomycin C [Uyeda et al., J Enzyme Inhib, 6(2):157-64 (1992)]; 5923-DNI [Uyeda et al, supra (1992)]; and pyridoxal 5′-phosphate, an inhibitor of ATP-dependent DNAse from Bacillus laterosporus [Fujiyoshi et al., J Biochem, (Tokyo) 89(4):1137-42 (April 1981)].

Other direct competitive nuclease inhibitors include polyclonal antibodies capable of binding to nucleases or fragments thereof [Crespeau et al., C R Acad Sci III, 317(9):819-23 (Sep 1994)]. Such antibody preparations are available from a variety of commercial sources including but not limited to Genentech, Worthington laboratories, and Sigma-Aldrich. Such antibodies can be prepared by immunizing a mammal with a preparation of a nuclease. Methods for accomplishing such immunizations are well known in the art. Monoclonal antibodies (or fragments thereof) can also be produced by immunizing splenocytes with activated APF (by modifying the procedures of Kohler et al. Nature 256:495 (1975); Eur. J. Immunol., 6:511 (1976); Euro J. Immunol., 6:292 (1976)]. Antibody-based DNA inhibitors can be adapted to incorporate human protein sequences (‘humanized antibody’) and thereby be used in humans without generating an immune response.

In a preferred embodiment, the nuclease inhibitor is ATA or a functional derivative thereof. ATA is a triphenylmethane-derivitive dye first synthesized in 1892, and initially prepared as a pure compound in 1949. The molecular mass of the polycarboxylated ATA molecule is 473 daltons, and it can polymerize into larger complexes of up to 6,000 Da [Guo et al., Thromb Res 71(1):77-88 (1993)]. It has been reported that ATA does not permeate intact cell membranes [Apirion et al., Springer-Verlag, 3:327-340 (1975)], and so presumably either must bind to extracellular factors such as nucleases or interact with cell membrane-associated molecules thereby altering intracellular events.

ATA inhibits many endonucleases including DNAse I, RNAse A, SI nuclease, exonuclease III, and a variety of restriction endonucleases [Hallick et al., Nucleic Acids Res, 4(9):3055-64 (1977)]. ATA-mediated inhibition of pancreatic ribonuclease A/nucleic acid complex formation has been investigated by proton magnetic resonance spectroscopy, and these experiments indicate that the mechanism of RNAse inhibition involves competition between the nucleic acid and polymeric ATA for binding in the active site of the protein [Gonzalez et al., Biochemistry, 19(18):4299-4303 (1980)]. In addition to inhibiting nuclease activity in vitro, many investigators have also employed the agent to inhibit apoptosis in cultured cells and tissues in vivo.

The anti-apoptotic activity of ATA is somewhat controversial, in part due to the wide variety of activities attributed to the compound. These activities include inhibition of topoisomerase II [Catchpoole et al., Anticancer Res, 14(3A):853-856 (1994)], induction of erbB4 phosphorylation [Okada et al., Biochem Biophys Res Commun, 230(2):266-269 (1997)], activation of the Jak2-Stat5 signaling athway [Rui et al., Biol Chem, 273(5273):352-354 (1998)], and inhibition of mu- and m-calpain activities [Posner et al., Biochem Mol Biol Int, 1995]. Topoisomerase II mediates chromatin condensation during apoptosis, and ATA inhibits the action of the protein at about 0.2 micromolar concentrations, levels lower than those usually employed to inhibit apoptosis.

Clearly, ATA acts to inhibit extracellular nuclease activity, and apparently also acts to inhibit various pathways associated with apoptosis. As both mechanisms can impact on the levels of reporter gene expression observed after direct administration of free plasmid, augmentation of reporter gene expression by ATA co-administration may be mediated by either or both mechanisms. Therefore, ATA can augment direct transfection of tissues by nuclease inhibition and/or by inhibition of apoptosis.

In addition to ATA itself, the invention contemplates the use of functional derivatives of ATA. A ‘functional derivative’ of ATA is a compound which possesses a biological activity (either functional or structural) that is substantially similar to a biological activity of ATA, for example inhibiting the activity of endogenous nucleases and/or inhibiting apoptosis. The term ‘functional derivative’ is intended to include the ‘fragments,’ ‘variants,’ ‘analogues,’ or ‘chemical derivatives’ of the compound. A ‘fragment’ of a compound such as ATA is meant to refer to any chemical subset of the molecule. A ‘variant’ of a compound such as ATA is meant to refer to a compound substantially similar in structure and function to either the entire compound, or to a fragment thereof. A compound is said to be ‘substantially similar’ to another compound if both compounds have substantially similar structures or if both compounds possess a similar biological activity. Thus, provided that two compounds possess a similar activity, they are considered variants as that term is used herein even if the structure of one of the compounds is not found in the other An ‘analogue’ or agent which mimics the function of a compound such as ATA is meant to refer to a compound substantially similar in function but not in structure to either the entire compound or to a fragment thereof. As used herein, a compound is said to be a ‘chemical derivative’ of another molecule when it contains additional chemical moieties not normally a part of the compound. Such moieties may improve the compound's solubility, absorption, biological half life, etc. The moieties may alternatively decrease the toxicity of the molecule, eliminate or attenuate any undesirable side effect of the molecule, etc. Moieties capable of mediating such effects are disclosed in Remington 's Pharmaceutical Sciences (1980). Procedures for coupling such moieties to a molecule are well known in the art. Analogues of ATA or agents which mimic the function of ATA can be used as transfection enhancing agents as well, inhibiting the activity of endogenous nucleases or apoptosis.

B. ‘Free’ Polynucleotide Preparations

The polynucleotide preparations of the instant invention are referred to as ‘free’ or ‘naked’. ‘Free polynucleotide(s)’ refers to DNA or RNA and can include sense and antisense strands as appropriate to the goals of the therapy practiced according to the invention. Polynucleotide in this context may include oligonucleotides and ribozymes. ‘Free’ in this context means polynucleotides which are not complexed to colloidal materials (including liposomal preparations), or contained within a vector which would cause integration of the polynucleotide into the host genome. As the free polynucleotide preparations of the instant invention are intended for direct delivery to target tissue, additional vector and vehicle components are not required. The free polynucleotide preparation may comprise one or more expression cassette and necessarily includes at least one polynucleotide of interest. In a preferred embodiment, the polynucleotide of interest is operably linked to the requisite transcription and translation elements required for expression of the polynucleotide of interest in eukaryotic organisms in situ.

Normally, an expression cassette is composed of a promoter region, a transcriptional initiation site, a ribosome binding site (RBS), an open reading frame (orf) encoding a protein (or fragment thereof), with or without sites for RNA splicing (only in eukaryotes), a translational stop codon, a transcriptional terminator and post-transcriptional poly-adenosine processing sites (only in eukaryotes) (Wormington, Curr. Opin. Cell Biol., 5:950-954 (1993); Reznikoff et al, Maximizing Gene Expression, Eds., Butterworths, Stoneham, Mass. (1986)).

The particular expression cassette employed in the present invention is not critical thereto, and can be selected from the many commercially available cassettes. Examples include but are not limited to pCEP4 and pRc/RSV (Invitrogen Corporation, San Diego, Calif.); pXT1, pSG5, pPbac and pMbac (Stratagene, La Jolla, Calif.); pPUR, pEGFP-1, pND and pMAM (ClonTech, Palo Alto, Calif.); and pSVβ-gal (Promega Corporation, Madison, Wis.). Alternatively, the cassette may be synthesized either de novo or by adaptation of a publicly or commercially available expression system.

When testing the invention in an experimental model, it may desirable to include a reporter gene in the expression cassette. Examples of reporter genes include but are not limited to beta-galactosidase, green fluorescent protein, and luciferase. An enhanced green fluorescent protein gene is commercially available and can be amplified from a commercial vector (pEGFP-1, Clonetech, Palo Alto, Calif.) incorporating Sal I and BamH I sites into the primers. The first 28 amino acids of the protein are from Drosophila Alcohol Dehydrogenase followed by the fused E. coli β-galactosidase sequences. The insect sequences are reported to give higher expression in mammalian cells presumably by providing eukaryotic translation initiation signals.

The individual elements within the expression cassette can be derived from multiple sources and may be selected to confer specificity in sites of action or longevity of the cassettes in the host cell or target tissue. Such manipulation of the expression cassette can be done by any standard molecular biology approach.

These expression cassettes usually are in the form of plasmids, and contain various promoters well-known to be useful for driving expression of genes in animal cells, such as the viral derived SV40, CMV and, RSV promoters or eukaryotic derived β-casein, uteroglobin, β-actin or tyrosinase promoters. The particular promoter is not critical to the present invention, except in the case where the object is to obtain expression in only targeted cell types or tissues. In a preferred embodiment, the promoter is selected to be one which is only active in the targeted cell type or tissue. Examples of tissue specific promoters include, but are not limited to the tyrosinase promoter which is active in lung and spleen cells, but not testes, brain, heart, liver or kidney [Vile et al, Canc. Res., 54:6228-6234 (1994)]; the involucerin promoter which is only active in differentiating keratinocytes of the squamous epithelia [Carroll et al, J. Cell Sci., 103:925-930 (1992)]; and the uteroglobin promoter which is active in lung and endometrium [Helftenbein et al, Annal. N.Y. Acad. Sci., 622:69-79 (1991)].

Alternatively, tissue/cell specific enhancer sequences can be used to control expression. Yet another way to control tissue specific expression is to use a hormone responsive element (HRE) to specify which cell lineages a promoter will be active in, for example, the MMTV promoter requires the binding of a hormone receptor, such as progesterone receptor, to an upstream HRE before it is activated [Beato, FASEB J., 5:2044-2051 (1991); and Truss et al, J. Steroid Biochem. Mol. Biol., 41:241-248 (1992)].

Additional genetic elements may be included on the expression cassette in order to modify its behavior inside the host cell [Hodgson, Bio/Technology, 13:222-225 (1995)]. Such elements include viral genome components such as the DNA genome of a recombinant adenovirus or the self-replicating “replicon” RNA of an alphavirus such as semliki forest or sindbus virus. Additional elements include but are not limited to mammalian artificial chromosome elements or elements from the autonomous replicating circular minichromosomes, such as found in DiFi colorectal cancer cells, to allow stable non-integrated retention of the expression cassette [Huxley et al, Bio/Technology. 12:586-590 (1994); and Untawale et al, Canc. Res., 53:1630-1636 (1993)], intergrase to direct integration of the expression cassette into the recipient cells chromosome [Bushman, Proc. Natl. Acad. Sci., USA, 91:9233-9237 (1994)], the inverted repeats from adeno-associated virus to promote non-homologous integration into the recipient cells chromosome [Goodman et al, Blood, 84:1492-1500 (1994)], recA or a restriction enzyme to promote homologous recombination [PCT Patent Publication No. WO9322443 (1993); and PCT Patent Publication No. WO9323534-A (1993)] or elements that direct nuclear targeting of the eukaryotic expression cassette [Hodgson, supra].

These additional genetic elements may also include substantial regions of viral genomes, so that integration and/or autonomous replication of the polynucleotide of interest will be enabled by the viral sequence elements. For example, inclusion of the AAV ITR sequences together with the rep protein ORF in the expression cassette can provide integration.

C. Administration, Dosage and Formulation

The instant invention focuses on direct polynucleotide delivery. This technique is often referred to as naked DNA injection, direct injection or free DNA injection. However, the administration route is not critical to the instant invention. The approach generally involves the introduction of a polynucleotide preparation encoding a polynucleotide of interest which is then expressed within cells of the host organism. In a preferred embodiment, the instant invention involves the direct application of high concentration polynucleotide preparations to target tissues. The findings of the instant invention are particularly applicable to those techniques utilizing naked or free polynucleotide. Parameters such as formulation, dosage and delivery means are all standardly optimized using routine techniques and are well within the purview of those skilled in the art.

Delivery of polynucleotides to animal tissues in vivo can be achieved by dermal administration, intramuscular injection, transmucosal and transepithelial delivery. The preferred routes of admiristration to the respiratory tract will be by inhalation or insufflation. Routes of administration to other mucosal tissues will vary according to their location. The parenteral routes of administration is possible in limited cases though not generally preferred as the crux of free polynucleotide delivery is minimal invasivity. The delivery technique (in vitro or in vivo) may involve direct injection, particle bombardment, jet injection systems, electroporation and cationic liposomes. The use of liposomes for delivery of the free polynucleotides of the invention is not preferred as such delivery mechanisms frequently result in reduced levels of expression.

‘Dermal’ administration refers to routes of administration which apply the free polynucleotide(s) on, to or through skin. Dermal routes include epidermal, intradermal and subcutaneous injections as well as transdermal transmission. The transdermal transmission may be active (e.g. delivery driven by iontophoresis) or passive (e.g., delivery driven by diffusion alone). Iontophoretic transmission may be accomplished using commercially available “patches” which deliver their product continuously through unbroken skin for periods of several days or more. Use of this method allows for controlled transmission of pharmaceutical compositions in relatively great concentrations, permits infusion of combination drugs and allows for contemporaneous use of an absorption promoter.

The introduction of the polynucleotide preparation can be accomplished by simple intramuscular (IM) or intradermal (D) injections using needles, the combination of needle or other injection methods together with electroporation, as well as by propelling DNA-coated gold particles through various tissues, preferentially the dermis. Although only a limited number of cells can be transfected using these methods, the level of expression of the polynucleotide of interest leads to surprisingly strong immune responses [Fynan E F et al., Proc Natl Acad Sci USA, 90(24):11478-11482 (1993); Davis H L et al., editor, Molecular and Cell Biology of Human Gene Therapeutics. London, Chapman and Hall (1995)].

The mucosal and systemic immune systems are compartmentalized (Mesteky, J. Clin. Immunol., 7:265-270 (1987); Newby, In: Local Immune Response of the Gut, Boca Raton, CRC Press, Newby and Stocks Eds., pages 143-160 (1984); and Pascual et al., Immuno. Methods., 5:56-72 (1994)). Thus, antigens delivered to mucosal surfaces elicit mucosal and systemic responses, whereas parentally delivered antigens elicit mainly systemic responses but only stimulate poor mucosal responses (Mesteky, supra). Moreover, mucosal stimulation at one mucosal site (for example the intestine) can result in development of immunity at other mucosal surfaces (for example genital/urinary tract) (Mesteky, supra). This phenomenon is referred to as the common mucosal system and is well-documented (Mesteky, supra; and Pascual et al, supra).

In the past, delivery of DNA molecules to mucosal surfaces is inefficient due to the many natural host defenses found at these surfaces, such as the gastric barrier and nucleases in the gastrointestinal tract, and the thick mucous layer in the respiratory tract. The instant invention allows for direct delivery of polynucleotides to mucosal sites to be revisited. For mucosal administration, the means of introduction will vary according to the location of the point of entry. Particularly for immunization to and treatment of respiratory infections, intranasal administration means are most preferred. These means include inhalation of aerosol suspensions or insufflation of the naked polynucleotide or mixtures thereof. Suppositories and topical preparations will also be suitable for introduction to certain mucosa, such as genital and ocular sites. Also of particular interest with respect to vaginal delivery of free polynucleotides are vaginal sandwich-type rings and pessaries.

Respiratory-associated epithelia may be accessed using drug delivery systems which are based on oro- and/or nasopharyngeal administration. Although the ontogeny, differentiation, function, and pathologies of cells lining respiratory tissues vary, this large, continuous, arborized epithelial surface shares the common feature of being exposed to inhaled gases and particulates. Physicians are able to select from a wide range of well developed technologies for administering drugs to this surface via the naso- and oropharynx. These technologies involve fluid formulations (nose drops, bronchoscopic instillation), traditional aerosols (sprays, inhalers, nebulizers), and particulates (dry powder aerosols). With the possible exception of submucosal glands and other specialized microenvironments, these established delivery technologies may be employed for genetic modification of virtually any respiratory-associated tissue once simple, safe, and effective methods for in vivo transfection/transduction of mucosae exist.

While opportunities for formulation development of viral and lipoplex-based polynucleotide delivery systems is constrained, there are many avenues for further advances in the formulation and administration of plasmid/nuclease inhibitor combinations. Slow release DNA depot preparations which are protected from nuclease activity, development of nonabsorbable nuclease inhibitors, and aerosol, dry powder, spray, and nose drop formulations may all be developed. The exceptional stability of double stranded plasmid relative to many other biopolymers, together with the small particle size of collapsed, supercoiled DNA, is compatible with many variations of these established delivery method themes.

Compositions of free polynucleotides and mixtures of polynucleotides may be placed into a pharmaceutically acceptable suspension, solution or emulsion. The particular pharmaceutically acceptable carrier or diluents employed is not critical to the present invention and will necessarily vary with the administration route, particular host organism and target tissue. For example, for intramuscular injection, the polynucleotide may be dissolved in water, saline or an endotoxin-free injectable PBS. The concentration preferably ranges from 0.1 to 2 mg/ml (w/v), more preferably 1 mg/ml (w/v). In an alternate embodiment, the polynucleotide may be injected in 25% (w/v) sucrose. Examples of diluents include a phosphate buffered saline, citrate buffer (pH 7.0) containing sucrose, bicarbonate buffer (pH 7.0) alone, or bicarbonate buffer (pH 7.0) containing ascorbic acid, lactose, and optionally aspartame. Examples of carriers include proteins, e.g., as found in slim milk, sugars e.g., sucrose, or polyvinylpyrrolidone (PVP). Typically these carriers would be used at a concentration of about 0.1-90% (w/v) but preferably at a range of 1-10% (w/v).

Suitable mediums include saline and may include liposomal preparations (for those embodiments which do not rely on antigen presenting cells for delivery of the polynucleotides into target tissue). More specifically, pharmaceutically acceptable carriers may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like. Further, a composition of free polynucleotides may be lyophilized using means well known in the art, for subsequent reconstitution and use according to the invention.

Where the free polynucleotides are to be introduced into skin or mucosa, delivery of the polynucleotide is preferably facilitated without need for injection by use of detergents, absorption promoters, chemical irritants (such as keratinolytic agents), or mechanical irritants. Detergents and absorption promoters which facilitate uptake of small molecules other than genes are well known in the art and may, without undue experimentation, be adapted for use in facilitating uptake of genes. Another substantially noninvasive approach to introducing the free polynucleotides is by transdermal transmission (preferably iontophoresis) which has been used with success for transdermal transmission of peptides.

The efficiency of the current system may be insufficient to enable correction of inborn genetic deficiencies. However, vaccine applications do not require efficient transfection and high levels of protein expression. Transfection or transduction of tissues associated with the posterior naso- and oropharynx enables development of mucosal immune responses to encoded antigens (see co-pending application Ser. No. 08/862,632 cited above, incorporated by reference in its entirety). Therefore genetic vaccination using nasal drop, spray, instillation or oropharyngeal aerosol formulations of plasmid/ATA mixtures enables priming and/or repetitive boosting of immune responses.

The amount and concentration of the polynucleotide to be administered will vary depending on the species of the subject, as well as the desired response and the disease or condition that is being treated. Generally, it is expected that up to 100-200 μg of DNA can be administered in a single dosage, although as little as about 0.3 μg of DNA administered through skin or mucosa can induce long lasting immune responses. For purposes of the invention, however, it is sufficient that the free polynucleotides be supplied at a dosage sufficient to cause expression of the polynucleotide of interest carried by the polynucleotide.

Dose-response experiments can be used to efficacy, toxicity, and effective dose of a particular enhancing agent or polynucleotide preparation. Such experiments were used to define an effective dose for ATA as an adjuvant for direct transfection of respiratory tissues. Histologic evaluation of treated lung tissue was subsequently performed. As virtually all medicines exhibit toxicity at some dose, an attempt was made to define the upper end of the dosing range by identifying a toxic dose for intratracheal ATA administration. ATA is often employed for inhibition of cultured cell apoptosis, but relatively few publications describe in vivo applications. In vivo dosing employed in published reports ranges from 4 micrograms administered to the night cerebral ventricle of gerbils to 10 mg/kg/hour continuous infusion in a hamster carotid stenosis model Based on this literature, a dose range from 0.1 mg/kg to 8 mg/kg ATA administered with 100 micrograms of plasmid was tested. In pilot experiments, 100% of mice treated with 8 mg/kg died within 8 to 24 hours, and 50%.of mice treated with 6 mg/kg died within the same time period. At 2 mg/kg ATA or lower doses, no lethality due to the ATA was observed during the 48 hours after treatment. Therefore, subsequent experiments were restricted to a dose range of 0.1 to 6 mg/kg ATA. Analysis of levels of luciferase reporter gene expression detected over this dose range identified a broad peak of enhancement which ranged from 0.3 to 4 mg/kg ATA, with a decline in enhancement at the 6 mg/kg dose. Subsequent histologic examination was performed using lung tissue obtained from mice treated with up to 5 mg/kg ATA.

D. Host Organism, Target Cell and Tissues

The polynucleotide preparations herein are specifically designed for direct delivery of the polynucleotide of interest to a particular host organism or target tissue or cell. The polynucleotide preparations may be used to enhance polynucleotide transfer to cells and/or tissues both in vivo and in vitro.

The target cell and/or tissue is not critical to the invention. The target tissues include all eukaryotic cells as well as prokaryotic cells in which intracellular nuclease activity reduces transfection activity. The cells may be present in the intact animal, a primary cell culture, explant culture or a transformed cell line. Certain cells or tissues contemplated by the instant invention include but are not limited to embryos and embryonic tissues, fetal tissues, oocytes, embryonic or pleuripotent tissue or cells. However, the particular cells and tissue source are of the cells not critical to the present invention. Injection of uncomplexed polynucleotides results in transfection of cells within a wide variety of tissues, including skeletal muscle, cardiac muscle, liver, tumors, skin, thyroid, thymus, synovium, and brain [Wolff, Nat Med, 3(8):849-854 (1990); Conry, Int J Biochem Cell Bio, 27:633-45 (1995); Acsadi, Biol Chem, 2(273):28-32 (1998); Malone, Science, 273(5273):352-354 (1996); Yang, Hum Gene Ther, 5(7):837-844 (1994); Raz, Science, 248:1019-1023 (1990); Sikes, Proc Assoc Am Physicians, 109(4):409-419(1997); Li, Hum Gene Ther, 8(7):817-825 (1997); Yovandich, Am J Physiol 268(6 Pt 1):1052-L1056 (1995)]. However, as direct delivery is required, the tissue to be targeted may be limited by access. New administration techniques are developed daily, it is foreseeable that tissues which are currently not available for direct delivery will be accessible at a later date using more current delivery technology.

The host organism employed in the present invention is not critical thereto and includes all organisms within the kingdom animalia, such as those of the families mammalia, pisces, avian, reptilia. Preferred animals are mammals, such as humans, bovines, ovines, porcines, felines, buffalos, canines, goats, equines, donkeys, deer, and primates. The most preferred animal is a human.

Within the host system, certain cells and tissues are preferred targets for expression of the polynucleotide of interest. As mentioned above, particular tissues of interest include but are not limited to skeletal muscle, cardiac muscle, vascular endothelium, liver, tumors, skin, thyroid, thymus, synovium, and brain. Likewise, in the context of the instant invention, preferred target tissues are those which have a high level of DNAse activity, those that secrete large amounts of DNAse, or those that exist in an environment having a high level of DNAse activity. Such tissues include but are not limited to hepatic, pancreatic, mucosal and respiratory tissues. Particular mucosal tissue or mucosal associated tissues contemplated include oral tissues, ocular tissues, gastro-intestinal tissues, gut-associated lymphoid tissues, bronchial-associated lymphoid tissues, nasal-associated lymphoid tissues, genital-associated lymphoid tissues, Waldeyer's ring, Peyer's patches, and tonsils. Particular respiratory tissues or respiratory associated tissues contemplated include oropharyngeal mucosa, nasopharyngeal mucosa, conducting airway epithelium, and pulmonary parenchyma. Respiratory tract tissues are subject to a variety of pathologies, provide a first line of defense against many environmental toxins and pathogens, are selectively permeable to a range of molecules including biopolymers, and are largely associated with a rich vascular bed. Therefore, technologies for genetic modification of respiratory tissues may be used to develop treatments for inborn errors of metabolism (such as Cystic Fibrosis), to treat systemic disease via absorption to or from the circulatory compartment or to modify either pathologic or beneficial immune responses (asthma, mucosal immunity).

E. The Polynucleotide of Interest

As used herein, ‘polynucleotide of interest’ means a polynucleotide sequence encoding a protein or fragment thereof or anti-sense RNA or catalytic RNA, which is endogenous or foreign to the particular host species. The term ‘recombinant polynucleotide’ refers to a polynucleotide of genomic, cDNA, semisynthetic or synthetic origin which is distinct in form, linkage or association from the form, linkage, or association in which the polynucleotide exists in nature. The term ‘recombinant DNA’ refers to a DNA molecule produced by operatively linking two DNA segments. Thus, a recombinant DNA molecule is a hybrid molecule comprising at least two nucleotide sequences not normally found together in nature. Recombinant DNA molecules not having a common biological origin (i.e., evolutionarily different) are said to be ‘heterologous’. Herein, the term ‘purified polynucleotide’ refers to a polynucleotide which is essentially free, i.e., containing less than about 50%, preferably less than about 70%, even more preferably less than about 90% of polypeptides with which the polynucleotide is naturally associated.

In the present invention, the polynucleotide preparation delivers a sequence specific polynucleotide of interest into a target cell or tissue. The polynucleotide of interest may encode for a gene, vaccine antigen, an immunoregulatory agent, or a therapeutic agent. The polynucleotide of interest may be either a foreign gene or a endogenous gene. The polynucleotide of interest need not encode a protein. Rather, the polynucleotide of interest may be a therapeutic polynucleotide or one which will affect the biology of a cell, tissue or host; such polynucleotides find particular utility in the field of gene discovery (e.g., identification and functional characterization of new genes) and rational drug design (e.g., identification and validation genetic targets for pharmaceutical manipulation). As used herein, ‘foreign gene’ means a polynucleotide encoding a protein or fragment thereof or anti-sense RNA or catalytic RNA, which is foreign to the recipient animal cell or tissue, such as a vaccine antigen, immunoregulatory agent, or therapeutic agent. An ‘endogenous gene’ means a polynucleotide encoding a protein or part thereof or anti-sense RNA or catalytic RNA which is naturally present in the recipient animal cell or tissue.

In the preferred embodiment, the polynucleotide of interest encodes a vaccine antigen. As used herein, the term ‘vaccine antigen’ refers to an agent capable of stimulating the immune system of a living organism, inducing the production of an increased level of antibodies, the production of a cellular immune response, or the activation other immune responsive cells involved in the immune response pathway against said antigen. The vaccine antigen expression may be performed to elicit an immune response and/or to induce tolerance to the encoded antigen. In particular, expression of antigens in cells which lack co-stimulatory molecule expression can enable the development of tolerance to the antigen.

The vaccine antigen may be a protein or antigenic fragment thereof from viral pathogens, bacterial pathogens, and parasitic pathogens. Alternatively, the vaccine antigen may be a synthetic polynucleotide, constructed using recombinant DNA methods, which encode antigens or parts thereof from viral, bacterial, parasitic pathogens. These pathogens can be infectious in humans, domestic animals or wild animal hosts. The antigen can be any molecule that is expressed by any viral, bacterial, parasitic pathogen prior to or during entry into, colonization of, or replication in their animal host.

The viral pathogens, from which the viral antigens are derived, include, but are not limited to, Orthomyxoviruses, such as influenza virus; Retroviruses, such as RSV and SIV, Herpesviruses, such as EBV; CMV or herpes simplex virus; Lentiviruses, such as human immunodeficiency virus; Rhabdoviruses, such as rabies; Picornoviruses, such as poliovirus; Poxviruses, such as vaccinia; Rotavirus; and Parvoviruses.

Examples of protective antigens of viral pathogens include the human immunodeficiency virus antigens Nef, p24, gp120, gp41, Tat, Rev, and Pol et al, Nature, 313:277-280 (1985)) and T cell and B cell epitopes of gp120(Palker et al, J. Immunol., 142:3612-3619 (1989)); the hepatitis B surface antigen (Wu et al, Proc. Natl. Acad. Sci., USA, 86:4726-4730 (1989)); rotavirus antigens, such as VP4 (Mackow et al, Proc. Natl. Acad. Sci., USA, 87:518-522 (1990)) and VP7 (Green et al, J. Virol., 62:1819-1823 (1988)), influenza virus antigens such as hemagglutinin or nucleoprotein (Robinson et al., Supra; Webster et al, Supra) and herpes simplex virus thyrnidine kinase (Whitley et al, In: New Generation Vaccines, pages 825-854).

The bacterial pathogens, from which the bacterial antigens are derived, include but are not limited to, Mycobacterium spp., Helicobacter pylori, Salmonella spp., Shigella spp., E. coli, Rickettsia spp., Listeria spp., Legionella pneumoniae, Pseudomonas spp., Vibrio spp., and Borellia burgdorferi.

Examples of protective antigens of bacterial pathogens include the Shigella sonnei form 1 antigen (Formal et al, Infect. Immun., 34:746-750 (1981)); the O-antigen of V. cholerae Inaba strain 569B (Forrest et al, J. Infect. Dis., 159:145-146 (1989); protective antigens of enterotoxigenic E. coli, such as the CFA/I fimbrial antigen (Yamamoto et al, Infect. Immun., 50:925-928 (1985)) and the nontoxic B-subunit of the heat-labile toxin (Clements et al, 46:564-569 (1984)); pertactin of Bordetella pertussis (Roberts et al, Vacc., 10:43-48 (1992)), adenylate cyclase-hemolysin of B. pertussis (Guiso et al, Micro. Path., 11:423-431 (1991)), and fragment C of tetanus toxin of Clostridium tetani (Fairweather et al, Infect. Immun., 58:1323-1326 (1990)).

The parasitic pathogens, from which the parasitic antigens are derived, include but are not limited to, Plasmodium spp., Trypanosome spp., Giardia spp., Boophilus spp., Babesia spp., Entamoeba spp., Eimeria spp., Leishmania spp., Schistosome spp., Brugia spp., Fascida spp., Dirofilaria spp., Wuchereria spp., and Onchocerea spp.

Examples of protective antigens of parasitic pathogens include the circumsporozoite antigens of Plasmodium spp. (Sadoff et al, Science, 240:336-337 (1988)), such as the circumsporozoite antigen of P. bergerii or the circumsporozoite antigen of P. falciparum; the merozoite surface antigen of Plasmodium spp. (Spetzler et al, Int. J. Pept. Prot. Res., 43:351-358 (1994)); the galactose specific lectin of Entamoeba histolytica (Mann et al, Proc. Natl. Acad. Sci., USA, 88:3248-3252 (1991)), gp63 of Leishmania spp. (Russell et al, J. Immunol., 140:1274-1278 (1988)), paramyosin of Brugia malayi (Li et al, Mol. Biochem. Parasitol., 49:315-323 (1991)), the triose-phosphate isomerase of Schistosoma mansoni (Shoemaker et al, Proc. Natl. Acad. Sci., USA, 89:1842-1846 (1992)); the secreted globin-like protein of Trichostrongylus colubriformis (Frenkel et al, Mol. Biochem. Parasitol., 50:27-36 (1992)); the glutathione-S-transferase's of Frasciola hepatica (Hillyer et al, Exp. Parasitol., 75:176-186 (1992)), Schistosoma bovis and S. japonicum (Bashir et al, Trop. Geog. Med., 46:255-258 (1994)); and KLH of Schistosoma bovis and S. japonicum (Bashir et al, supra).

In another embodiment of the invention, the polynucleotide of interest can encode a therapeutic agent to animal cells or animal tissue. For example, the polynucleotide can encode tumor-specific, transplant, or autoimmune antigens or parts thereof. Alternatively, the polynucleotide can encode synthetic genes, which encode tumor-specific, transplant, or autoimmune antigens or parts thereof.

Examples of tumor specific antigens include but are not limited to prostate specific antigen (Gattuso et al, Human Pathol., 26:123-126 (1995)), TAG-72 and CEA (Guadagni et al, Int. J. Biol. Markers, 9:53-60 (1994)), MAGE-1 and yrosinase (Coulie et al, J. Immunothera., 14:104-109 (1993)). Recently it has been shown in mice that immunization with non-malignant cells expressing a tumor antigen provides a vaccine effect, and also helps the animal mount an immune response to clear malignant tumor cells displaying the same antigen (Koeppen et al, Anal. N.Y. Acad. Sci., 690:244-255 (1993)).

Examples of transplant antigens include the CD3 receptor on T cells (Alegre et al, Digest. Dis. Sci., 40:58-64 (1995)). Treatment with an antibody to CD3 receptor has been shown to rapidly clear circulating T cells and reverse most rejection episodes (Alegre et al, supra).

Examples of autoimmune antigens include IAS β-chain (Topham et al, Proc. Natl. Acad. Sci., USA, 91:8005-8009 (1994)). Vaccination of mice with an 18 amino acid peptide from IAS β-chain has been demonstrated to provide protection and treatment to mice with experimental autoimmnune encephalomyelitis (Topham et al, supra).

In an alternate embodiment of the present invention, the polynucleotide of interest can encode immunoregulatory molecules. These immunoregulatory molecules include, but are not limited to, growth factors, such as M-CSF, GM-CSF; and cytokines, such as IL-2, IL-4, IL-5, IL-6, IL-10, IL-12 or IFN-gamma. Delivery of cytolines expression cassettes to tumor tissue has been shown to stimulate potent systemic immunity and enhanced tumor antigen presentation without producing a systemic cytokine toxicity (Golumbek et al, Canc. Res., 53:5841-5844 (1993); Golumbek et al, Immun. Res., 12:183-192 (1993); Pardoll, Curr. Opin. Oncol., 4:1124-1129 (1992); and Pardoll, Curr. Opin. Immunol., 4:619-623 (1992)).

In an alterate embodiment of the present invention, the polynucleotide of interest may encode an antisense RNA or catalytic RNA. The RNA can be targeted against any molecule present within the recipient cell or likely to be present within the recipient cell. These include but are not limited to RNA species encoding cell regulatory molecules, such as interlukin-6 (Mahieu et al, Blood, 84:3758-3765 (1994)), oncogenes such as ras (Kashani-Sabet et al, Antisen. Res. Devel., 2:3-15 (1992)), causitive agents of cancer such as human papillomavirus (Steele et al, Canc. Res., 52:4706-4711 (1992)), enzymes, viral RNA's and pathogen derived RNA's such as HIV-1 (Meyer et al, Gene, 129:263-268 (1993); Chatterjee et al, Sci., 258:1485-1488 (1992); and Yamada et al, Virol., 205:121-126 (1994)). The RNAs can also be targeted at non-transcribed DNA sequences, such as promoter or enhancer regions, or to any other molecule present in the recipient cells, such as but not limited to, enzymes involved in DNA synthesis or tRNA molecules [Scadnlon et al, Proc. Natl. Acad. Sci. USA, 88:10591-10595 (1991); and Baier et al, Mol. Immunol., 31:923-932 (1994)].

In the experiments described in detail below, a plasmid was administered in combination with pharmaceuticals which indirectly or directly inhibit nucleases. It was observed that DNAse activity in lung fluids significantly contributes to the respiratory clearance of extracellular plasmid. A plasmid employing the human CMV IE promoter/enhancer to drive expression of the P. pyralis luciferase reporter protein was intratracheally administered into mouse lung +/− the nuclease/apoptosis inhibitor aurin tricarboxylic acid (ATA). Lavage samples and tissue extracts were used to characterize nuclease activity. ATA dose escalation experiments were performed using lung homogenate reporter protein assays to characterize transfection. Potential toxicity was assessed histologically. Reporter protein levels detected in lung extracts were markedly enhanced (50 to 65 fold) by treatment with the nuclease/apoptosis inhibitor aurin tricarboxylic acid (ATA), but not with EDTA or sodium citrate. Histological features of tissues treated +/− optimized ATA were indistinguishable. It is conceivable that the transfection enhancing activity of ATA involves inhibition of nuclease activity and/or interference with apoptosis. The identification and pharmaceutical inhibition of pathways which limit the tissue transfection activity of ‘free’ plasmid can enhance the efficiency of direct DNA delivery systems.

### EXAMPLES

The following examples are provided for illustrative purposes only, and are in no way intended to limit the scope of the present invention. The examples described herein address the problem of nuclease-mediated clearance of ‘free’ polynucleotides resulting in a significant reduction in the efficiency of direct DNA transfection of respiratory tissues. In vitro and in vivo assays were used to document DNAse activity in respiratory tissue fluids. To assess the functional significance of nuclease-mediated clearance, low molecular weight nuclease inhibitors were mixed and administered together with plasmid encoding the luciferase reporter protein. It was discovered that one can dramatically increase the efficiency while maintaining the safety of this simple polynucleotide delivery system by identifying and modulating biologic processes which influence the in vivo transfection activity of ‘free’ polynucleotides.

### Examples I

### In Vitro Experiments

The examples herein demonstrate the method of using ATA, a direct competitive nuclease inhibitor, to augment and increase the tissue transfection activity associated with direct administration of free polynucleotide.

1. Plasmids and Chemical Reagents

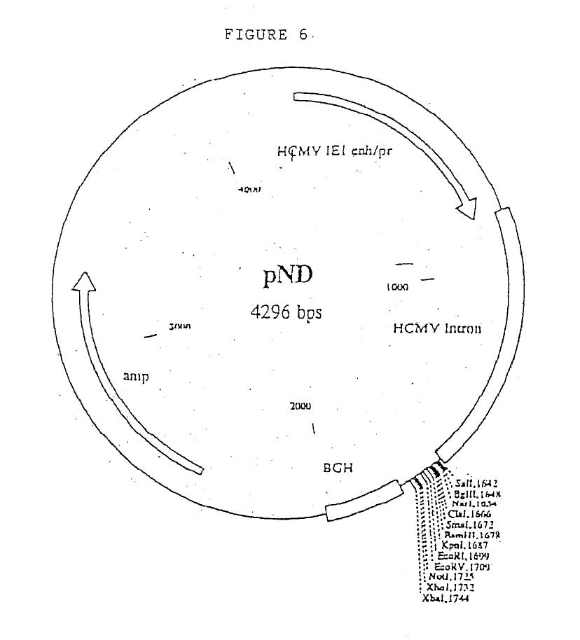

The P. pyralis luciferase cDNA was subcloned into the plasmid pND2/CMV. This plasmid was transformed into competent E. coli DH5-a cells, amplified in terrific broth, and prepared by alkaline lysis with the isolation of covalently closed circular plasmid DNA using two rounds of CsCl-EtBr gradient ultracentrifugation. The plasmid DNA was subsequently treated with DNAse-free RNAse, phenol/chloroform extracted, and purified by precipitation from an ethanol/sodium acetate solution. DNA purity was determined by agarose gel electrophoresis and optical density (OD 260/280 greater than or equal to 1.8). The resulting plasmid DNA is referred to as pND2Lux. See FIG. 6 for plasmid map.

DOTAP was prepared neat as a dry thin film (1 mM), followed by resuspension in sterile water (1 ml, 1 mM) with vortex mixing and sonication at the time of use. Aurin tricarbarboxylc acid (Sigma A-0885) was used to test the hypothesis that ATA might affect DNA transfection in both the in-vivo and in-vitro systems. The doses and treatment groups are described below.

The amplified DNA was inserted into pND using the same sites. The product was tested for activity by transfecting NIH 3T3 cells and staining for enzyme activity. The β-galactosidase Expression Vector (pNDbeta) may further be utilized. A hybrid β-galactosidase gene was amplified from a commercial vector (pCMVB, Clonetech, Palo Alto, Calif.) incorporating Sal I and BamH I sites into the primers. The first 28 amino acids are from Drosophila Alcohol Dehydrogenase followed by the fused E. coli β-galactosidase sequences. The insect sequences are reported to give higher expression in mammalian cells presumably by providing eukaryotic translation initiation signals. The amplified DNA was inserted into pND using the same sites. The product was tested for activity by transfecting NIH 3T3 cells and staining for enzyme activity.

2. Cell Culture

The human respiratory epithelial cell line, 16HBE14o (Dieter Grunert, UCSF) was cultured on flasks coated with fibronectin (Collaborative Research-Bectin Dickinson), vitrogen (Collagen Corp), basal media (Biofluids, Inc.) and bovine serum albumen (Biofluids, Inc.) as previously described [Gruenert, 1990 #7; Cozens, 1994 #8]. Cells were cultured on twenty four well tissue culture plates (Corning) coated with the above mixture. Culture media was Eagle's modified essential medium (EM) supplemented with 10% fetal bovine serum. 16HBE 14o cells were plated at a density of 5×10⁴ cells/ml 24 hours prior to transfection and cells were transfected as subconfluent monolayer.

NIH3T3 (ATCC CRL 1658) were grown in Dulbecco's Modified Eagle's Medium and 10% calf serum. NIH3T3 were also plated at a density of 5×10⁴ cells/ml in 24 well plates 24 hours prior to transfection and cells were transfected as subconfluent monolayer.

3. Transfection of Cultured Cells

The sequential addition of appropriate amounts of serum-free DMEM, plasmid DNA (1 mg/well), ATA in serum-free DEM and filter sterilized (see below for doses), and DOTAP (DOTAP:DNA formulation of 5 μg: 1 μg) were pippetted into a 2 ml Eppendorf tube to give a total volume of 800 ml and mixed with a vortex mixer. A 200 ml aliquot of the resultant transfection complex was added to each well of the cells above after the cell media had been aspirated off. The cells were incubated for 2 hours at 37° C. At the two hour time point, the mixture was removed and one ml of culture media was added to each well. Free DNA and DNA with the addition of ATA were used as controls.

The treatment groups of aurintricarbarboxyic acid were 80 μg/ml, 40 μg/ml, 10 μg/ml, 2 μg/ml, 1 μg/ml, 0.5 μg/ml, 0.25 μg/ml, and 0.0125 μg/ml. Each treatment group was repeated in four wells. All experiments were repeated twice and both NIH3T3 and 16HBE14o cell lines were used.

In vitro transfections using NIH3T3 and 16HBE14o cell lines yielded no significant differences between groups that received the DNA with ATA/DOTAP versus those groups that just received the DNA/DOTAP complexes at ATA does which were comparable to those given to mice. However, at the very highest doses (80 μg/ml, 40 μg/ml, 10 μg/ml), the ATA was toxic and killed cells which greatly reduced the number of relative light units.

The examples described herein shows a 57% increase over free DNA transfections with doses of ATA that were virtually nontoxic to the lung as shown by H&E staining. Health assessments were also used to determine the health status mice for up to 48 hours after transfection. All mice were healthy with no physical or behavioral side effects observed. The observation of surprising levels of luciferase reporter protein expression after direct application of plasmid/ATA mixtures to mouse lung tissue opens the door to a number of opportunities for further development of direct transfection of respiratory tissues, and may have implications for other polynucleotide delivery systems.

4. Comparison Of Transfection Enhancing Activity

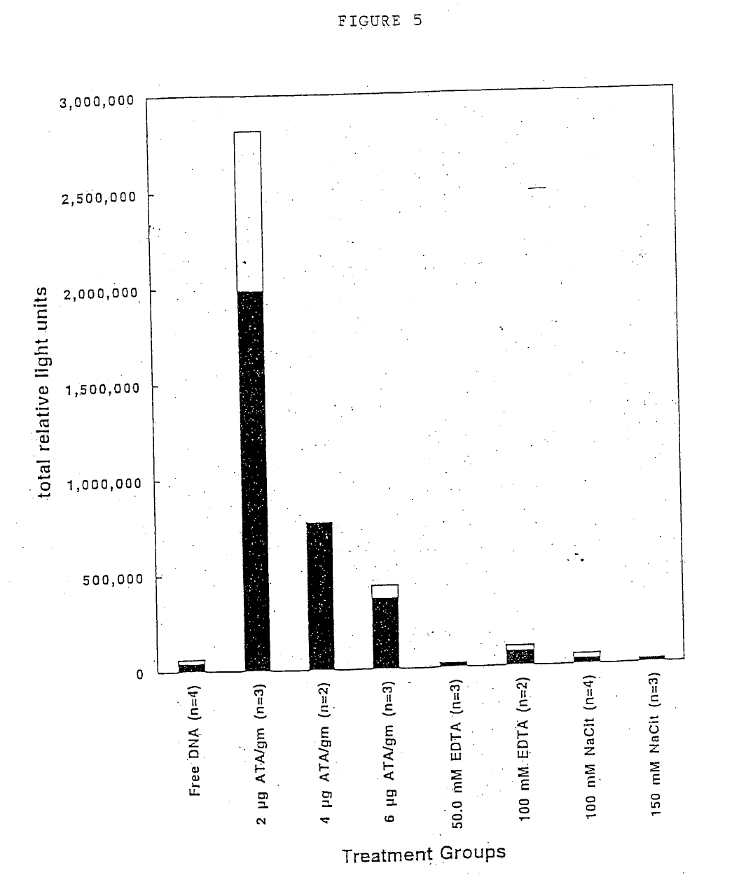

To determine if the ‘transfection enhancing ’ activity associated with ATA co-administration with free plasmid represents a general property of all DNAse inhibitors, comparative treatment experiments were performed with the divalent cation chelators EDTA, and sodium citrate. As summarized in FIG. 5, ATA co-administration markedly augmented the levels of reporter protein expression detected after intratracheal administration of plasmid. Neither EDTA nor citrate significantly enhanced the level of expression. In fact, administration with EDTA was associated with significant toxicity.

5. Nuclease Activity in Lung Lavage Fluid

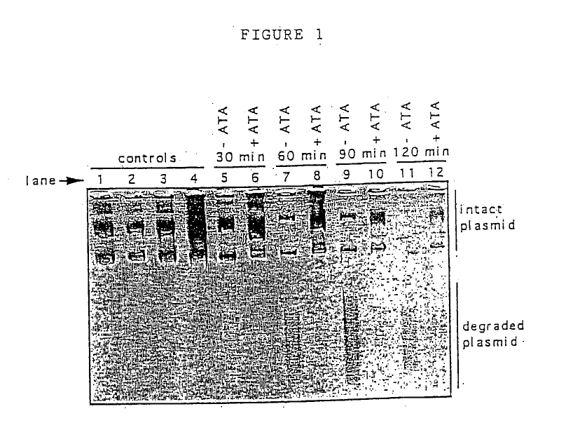

To determine if murine intrapulmonary fluids are associated with significant levels of nuclease activity, mice were sacrificed and the lungs were lavaged with 300 microliters of phosphate buffered saline. 10 microliters of this saline wash was then mixed with 750 ng of plasmid DNA in PBS to yield a total of 20 microliters. Samples were incubated for ½ hour to 2 hours at 37° C., and 1% minigel agarose TBE gel electrophoresis performed. The data summarized in the FIG. 1 is representative of assays performed using lavage fluid obtained from 4 different mice, and demonstrates the presence of significant levels of nuclease activity in murine lung lavage fluid.

6. ATA-Mediated Inhibition of Nuclease Activity

To detect degradation of intratracheally administered plasmid, and to assess the effect of ATA on this process in vitro, lavage samples were prepared as described above, and a whole lung tissue extract was prepared by homogenization of lung in PBS followed by clarification via centrifugation. As a positive control for DNAse activity, dornase alpha (Pulmozyrne, Genentech, So. SF, CA) was obtained as a 1 mg/ml solution. Optimal conditions for degradation of plasmid were determined by mini gel analysis. The treatment protocol described in Table c2 was then performed with either lavage fluid (FIG. 2A, incubation 2 hours 37C) or tissue extract (FIG. 2B, incubation ½ hour, 37C). Electrophoresis was performed in the absence of ethidium bromide, and plasmid runs in the order: form 1 (linear) fastest, form 2 (nicked circle) intermediate, form 3 (supercoiled) slow. The data demonstrate the presence of DNAase activity in both lavage fluid (less activity) and homogenates (more activity), the preferential degradation of form 1, and the inhibition of both pulmozyme and pulmonary-derived DNAse activity by ATA. See Table 1 below for discussion of the in vitro lung nuclease tests used.

TABLE 1
Protocol for in vitro lung nuclease tests used for FIGS. 2A and 2B
Experimental conditions:
(1) DNA used = pND2 Lox (289 ng/microliter);
(2) dosage pulmozyme = 1 mg/ml;
(3) concentration ATA = 1 mg/ml in water;
(4) concentration EDTA = 0.5 M
Results:

| | RNAse | PBS | DNA | ATA | Lavage Fluid | |
|---|---|---|---|---|---|---|
| lane | (in μl) | (in μl) | (in μl) | (in μl) | (in μl) | Pulmozyme |
| 1 | 2 | 13.4 | 2.6 | — | — | 2 μl |
| 2 | 2 | 13.4 | 2.6 | — | — | 2 μl 1:10 |
| | | | | | | dil |
| 3 | 2 | 13.4 | 2.6 | — | — | 2 μl 1:100 |
| | | | | | | dil |
| 4 | 2 | 11.4 | 2.6 | 2 | — | 2 μl |
| 5 | 2 | 11.4 | 2.6 | 2 | — | 2 μl 1:10 |
| | | | | | | dil |
| 6 | 2 | 11.4 | 2.6 | 2 | — | 2 μl 1:100 |
| | | | | | | dil |
| 7 | 2 | 11.4 | 2.6 | — | 10 | — |
| 8 | 2 | 7.4 | 2.6 | — | 8 | — |
| 9 | 2 | 5.4 | 2.6 | — | 4 | — |
| 10 | 2 | 9.4 | 2.6 | 2 | 10 | — |
| 11 | 2 | 5.4 | 2.6 | 2 | 8 | — |
| 12 | 2 | 3.4 | 2.6 | 2 | 4 | — |
| 13 | 2 | 8.4 | 2.6 | 3 | 4 | — |
| 14 | 2 | 7.4 | 2.6 | 4 | 4 | — |
| 15 | 2 | 5.4 | 2.6 | 6 | 4 | — |
| 16 | 2 | 13.4 | 2.6 | 2 | — | — |
| 17 | 2 | 9.4 | 2.6 | 6 | — | — |
| 18 | 2 | 15.4 | 2.6 | — | — | — |
| — | — | — | — | EDTA | — | — |
| | | | | (in μl) | | |
| 19 | 2 | 11.4 | 2.6 | 2 | — | 2 μl 1:10 |
| | | | | | | dil |
| 20 | 2 | 3.4 | 2.6 | 2 | 10 | — |

### Example II

### In Vivo Transfection—Mice

Five 7 week old female Balb-C mice (Charles River) were used for the in vivo experiments. All mice were anesthetized with a mixture of ketamine (22 mg/kg), xylazine (2.5 mg/kg) and acepromazine 0.75 mg/kg) IP prior to both intratracheal or intramuscular (IM) injections.

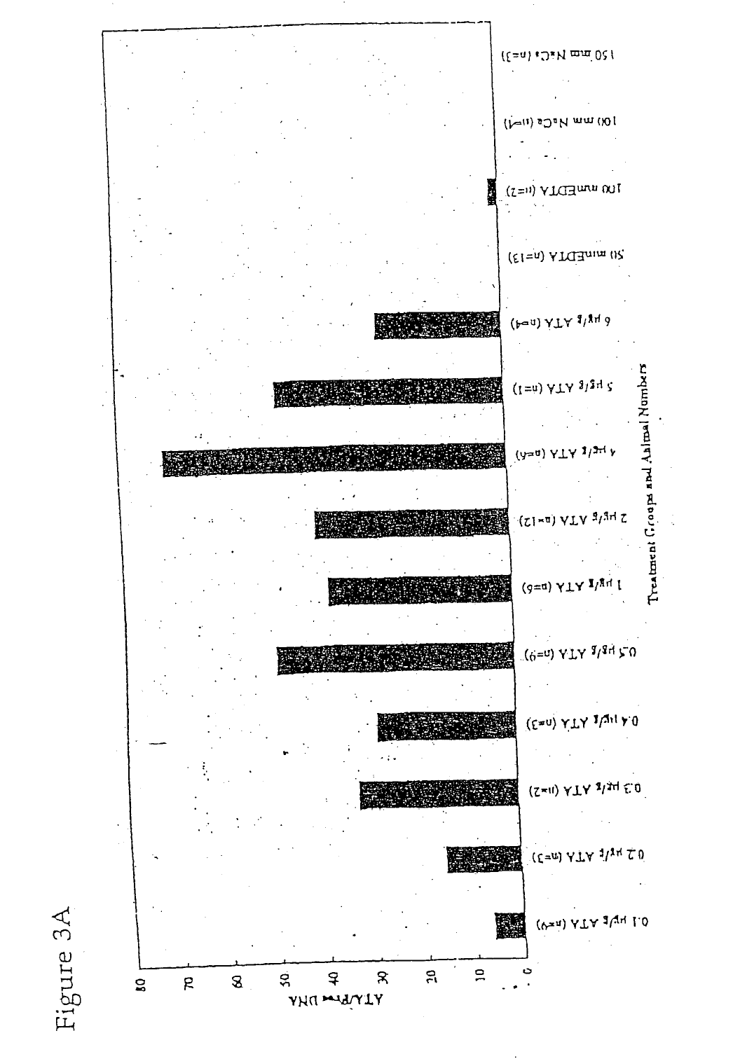

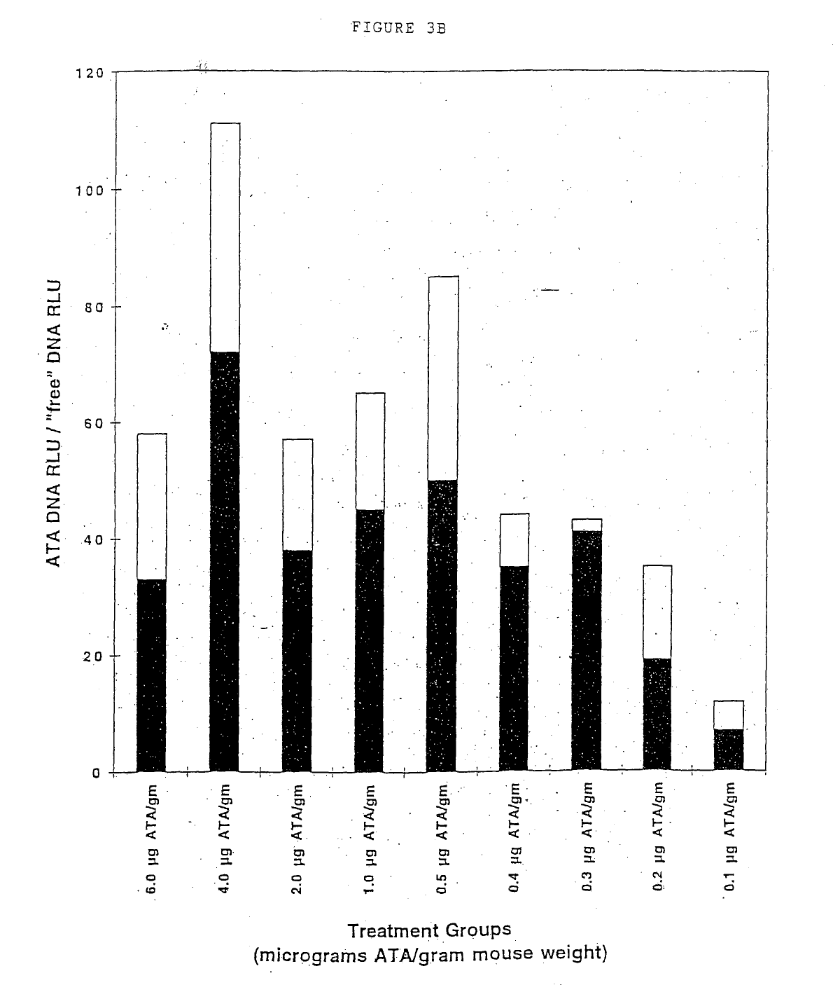

Intratracheal injections were performed by making a 1 cm medial cut through the skin at the ventral site of the neck. The musculature and salivary gland were teased apart using blunt dissection to expose the trachea. With the trachea visualized, a 30.5 gauge needle with a 1 cc tuberculin syringe was placed through the rings of the trachea toward the bronchi. DNA and DNAse inhibitors with DNAse inhibitors (see below) for a total volume of 100 μl in water for injection were administered into the lung. After injection, the salivary gland was placed back over the trachea, and the superficial neck wound was closed with staples using a 9 mm autoclip applier (Bectin-Dickinson). Intratracheal treatment groups consisted of four mice and each group received the following treatments: 8 μg/g, 6 μg/g, 4 μg/g, 2 μg/g, 1 μg/g, 0.5 μg/g, 0.4 μg/g, 0.3 μg/g, 0.2 μg/g, or 0.1 μg/g of ATA, 50 mm or 100 mm EDTA, 50 mm or 100 mm sodium citrate with the 100 μg of DNA. All experiments had a DNA alone group and water for injection only as a control group. All experiments were repeated at least once. The results are shown in FIGS. 3A and 3B.

Intramuscular injections were performed by injecting 50 μl of DNA or DNA/ATA complexes into the quadriceps muscle. Intramuscular treatment groups consisted of four mice. All groups received pND2Lux with or without 6 μg/g ATA.

Mice were euthanized using CO2 at 48 hours and the tracheal lung block or quadriceps muscle were dissected out, and assayed as described below.

1. Optimization Of Efficacy Of ATA/Plasmid Administration

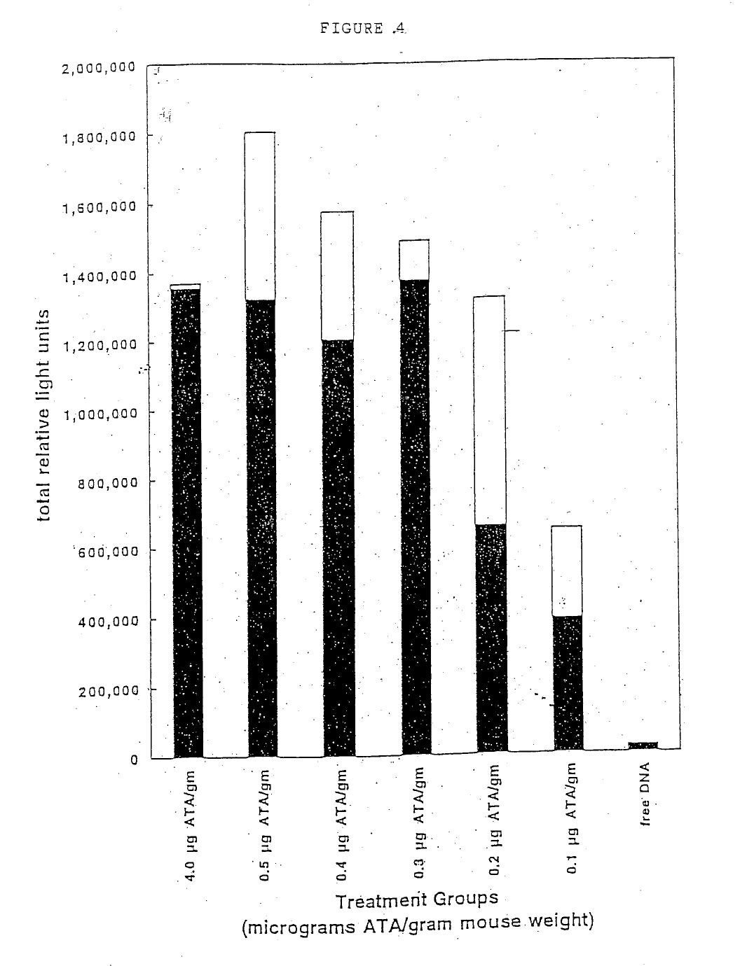

To provide a standard for comparison, one representative experiment from the set summarized in FIGS. 3A and 3B is included in FIG. 4. This data provides a mean and standard deviation of total relative light units (RLUs) less background obtained from groups of three similarly aged animals treated under the same conditions with same DNA at the same tine. Each mouse was treated with 100 micrograms of pND2Lux in 100 microliters of water via intratracheal installation using the methods described in detail below.

2. Luciferase Assay

Relative luciferase activity was determined using the Enhanced Luciferase Assay Kit and Monolight 2010 luminometer (Analytical Luminescence Laboratories, San Diego, Calif.). For tissue culture, this was accomplished by directly applying 330 μl of concentrated luciferase lysis buffer to each well and placing the cells on ice for 30 minutes. For in vivo experiments, each lung/trachea block or quadricep muscle was homogenized in 1 ml of lysis buffer (diluted 1:3 in distilled water). Samples were allowed to sit on wet ice for thirty minutes. For In vitro experiments, luciferase light emissions from 20 μl of the cell lysate were measured over a 10 second period, and results expressed as a function of the total lysate volume. For in vivo experiments, luciferase light emissions from 40 μl of the lysate were measured over a 10 second period, and results expressed as a function of the total lysate volume.

3. Histology

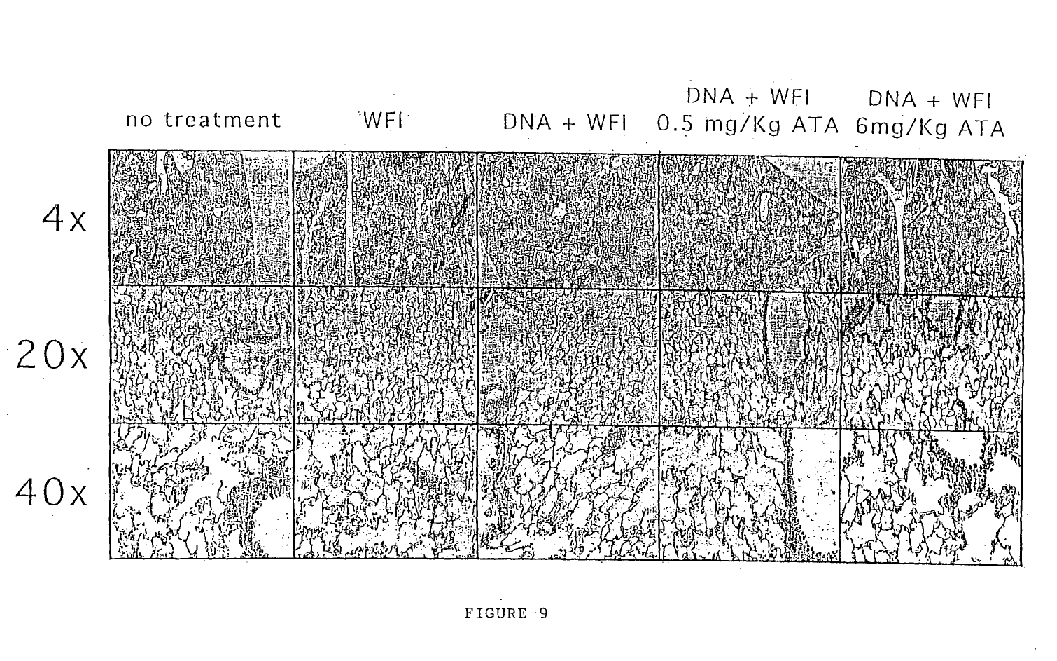

To obtain information about toxicity, mouse lung tissue was treated with ATA +/− plasmid and routine histologic analyses were performed. High pulmonary doses of ATA (>2 μg/g body weight) were associated with significant mortality, and expression of significant levels of TNF-α after intratracheal ‘free’ plasmid administration have been reported by Tsan et al Hum Gene Ther, 8(7):817-825 (1997). Therefore, intratracheal instillation of plasmid +/− ATA was performed, and two days later the treated tissues were histologically analyzed after inflation and fixation in formalin followed by routine hematoxylin and eosin staining in paraffin sections. Treatment groups included untreated controls, 100 μl of water for injection (WFI), 100 μg of pND2Lux diluted in WFI, and plasmid with 0.5 or 6 mg ATA/g body mass diluted in WFI. The images shown in FIG. 9 are representative photomicroscopic fields that include both conducting airway and pulmonary parenchyma. No significant histologic alterations were observed in the plasmid and ATA treated groups relative to the control treated lungs.

### Example III

### In Vivo Transfection—Macaque

Extending the experiment from murine to nonhuman primate respiratory tissues, the activity of ‘free’ plasmid DNA +/− ATA in macaque respiratory tissues using male rhesus macaques was tested. Initially, the lavage fluid is tested for DNAse activity +/− ATA by incubation with plasmid after which gel electrophoresis is performed as described above with murine lung lavage samples. After a 70 day quarantine, the animals are treated using three general techniques. For direct administration of lipid:DNA complexes to pulmonary parenchyma, macaques are anesthetized (ketamine, veterinarian assisted) and a pediatric bronchoscope will be passed per nasum with direct visualization of the bronchial tree. Typically, plasmid preparations (3 ml/Kg macaque weight) or related treatments (plasmid/ATA mixtures etc.) are instilled directly via the bronchoscope into the right lower lobe. The left upper lobe of the same macaque is used as an internal control. Animals are euthanized at appropriate times (typically 48 hours) and a necropsy performed. Typically, the lung and trachea are removed en block and dissections are performed to isolate pulmonary parenchyma and various generations of conducting airway. The tissue is then sampled for reporter gene assay and histologic analysis.

For nasal administrations, macaques are again anesthetized in a similar fashion and preparations are administered dropwise to the medial surface of the inferior nasal turbinate. In this case, it is not necessary to euthanize the animals, but rather tissue is punch biopsied and directly assayed for reporter gene expression (typically luciferase).

1. Comparison Of Cationic Lipid/DNA Complexes And ‘Free’ Plasmid

To assess the nonhuman primate pulmonary transfection activity of cationic lipid/DNA formulations optimized in mice, Journal of Liposome Research, 6(3):545-565 (1996) rhesus macaques (two groups of three each) were either treated with ATA/pND2Lux formulations or with a corresponding dose of ‘free’ plasmid. Treatments were performed via bronchoscopic administration as described herein. 240 micrograms of plasmid were administered in 8 ml water for injection, typically to the right lower lobe. Animals were necropsied 48 hours after treatment, lung was removed en bloc, dissection performed to obtain tracheal, primary, secondary and tertiary airway as well as pulmonary parenchyma. Tissue samples were either formalin fixed, processed and stained (H&E), were frozen in OCT for in situ hybridization, or were homogenized and assayed for luciferase activity. Typical levels of luciferase expression are summarized below in Table 2, and key findings from these experiments include 1) lack of histologically apparent acute toxicity with ‘free’ plasmid administration with or without ATA, and 2) substantially higher levels of luciferase activity in tissues treated with ‘free’ plasmid and ATA.

TABLE 2
Levels of luciferase expression found in each portion of the lung tissue sampled*

| DNA | | Left | Right | | LUL | RLL | RLL | RLL |
|---|---|---|---|---|---|---|---|---|
| administered | Trachea | Mainstream | Mainstream | LUL | Ad | Prox | Distal | Al |
| ‘Free DNA’ | — | — | — | — | — | — | — | — |
| J586 | 0 | 0 | 0 | 0 | 0 | 0 | 0 | 1539 |
| J626 | 0 | 0 | 0 | 129 | 0 | 0 | 16,313 | 19,583 |
| ‘DNA with | — | — | — | — | — | — | — | — |
| ATA’ | | | | | | | | |
| 11579 | 1140 | 1488 | 193 | 250 | 1,082,645 | 0 | 486,957 | 145,939 |
| AB85 | 1328 | 150 | 911 | | 0 | 10.438 | 282,388 | 549,810 |

(*values are relative light units or RLUs per 100 microliter sample)

2. Reporter Gene Assays

As described above, relative luciferase activity is determined using the Enhanced Luciferase Assay Kit and a Monolight 2010 luminometer (both from Analytical Luminescence Laboratories, San Diego, Calif.). Typically the tissue sample is weighed and 2 volumes of lx luciferase lysis buffer (final concentration 0.1M KPO₄pH 7.8, 1% Triton X-100, 1 mM DTT, 2 mM EDTA) is added and the sample is stored on water ice. Samples are then homogerized to uniformity using a Branson sonifier (typically 30 seconds to one minute). Luciferase light emissions from 31 microliters of the lysate are measured over a 10 second period, and results are expressed as a function of total protein, total tissue mass, and total DNA dose.

XGal histochemistry is performed for the detection of beta-galactosidase. Lung tissue is removed and briefly inflated using a solution of 2 g paraformaldehyde in 0.1M pipes buffer (30.2 g/l, pH=6.9) containing 2 mM MgCl₂ and 1.25 mM EGTA. The tissue is then be embedded in OCT and frozen in liquid nitrogen. The frozen tissue is then be cryo-sectioned (10 to 20 microns), placed on poly-lysine coated slides, re-fixed and then stained. Fixation of cryosections consists of treatment with paraformaldehyde/pipes (4C, 5 minutes) followed by washing (IX PBS with 2 mM MgCl₂, 5 minutes, 4C, repeat twice) and permeabilization (lx PBS, 2 mM MgCl₂, 0.01% sodium deoxycholate, 0.02% NP40, 5 minutes, 4C). The sections are then stained for 2 to 8 hours with Xgal (5-bromo-4-chloro-3-indoyl b-D-galactopyranoside, 1 mg/ml) in 30 mM KiFe(CN)₆, 30 mM K4Fe(CN)_{6.3}H₂O, 2 mM MgCl₂ 0.01% sodium deoxycholate, 0.02% NP40 in 1×PBS. (37C) After staining, the sections are again washed in PBS, lightly counter stained with eosin, and cover slipped for analysis.

Enhanced green fluorescent protein activity is assessed using wet tissue obtained either at necropsy or via biopsy. For routine analysis, wet tissue is compressed between glass slides and viewed with an inverted Nikon eclipse microscope (TE 200) equipped with 4, 10, 40 and 60× objectives and an appropriate light source and filters. Images are captured using-a Dage 330 3 chip color digital camera and a Scion CG-3 color capture board operated from a Macintosh 7200 under the control of Scion Image software (v1.62). This system enables frame averaging and compilation, providing substantial low light sensitivity.

### Example IV

### Intradermal Injection—Mouse, Rat, and Macaque

To further confirm the utility of nuclease inhibitors in combination with direct DNA delivery, the activity of free plasmid DNA +/− ATA in skin tissues was tested. The plasmid and animal protocols were identical to those described above. Intradermal injections were performed by injecting 100 μl solutions using 30 gauge needle.

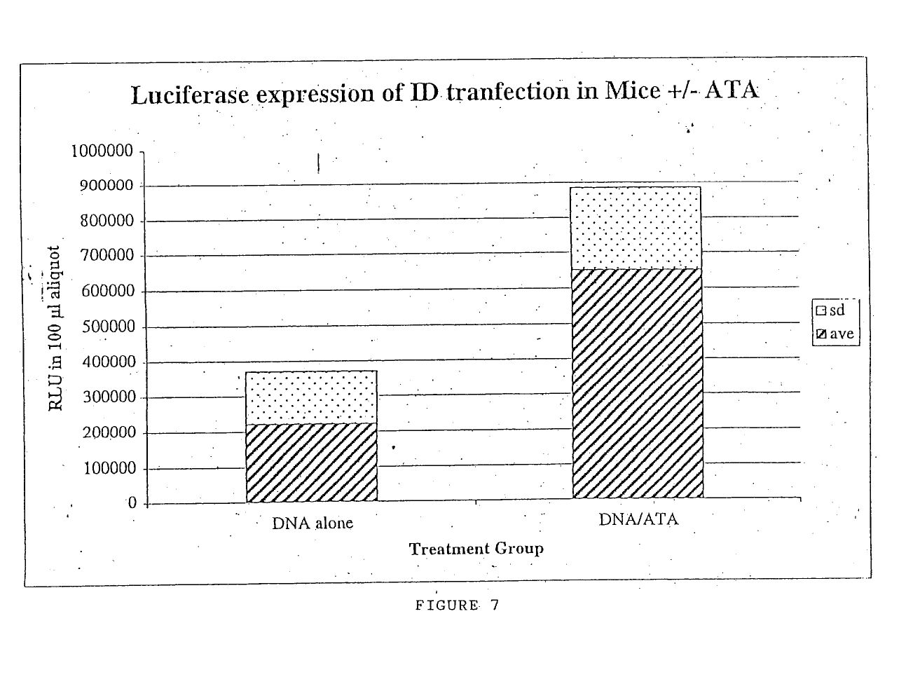

Intradermal injections were first performed using mice. The control solutions contained 100 μg of pND21ux plasmid in 100 μl water. The test solutions contained 100 μg of pND21ux plasmid and 20 μg of ATA in 100 μl water. Four mice were included in the control group and five mice were included in the test group. The results are shown in FIG. 7. Statistical analysis of the results shows p=0.0109.

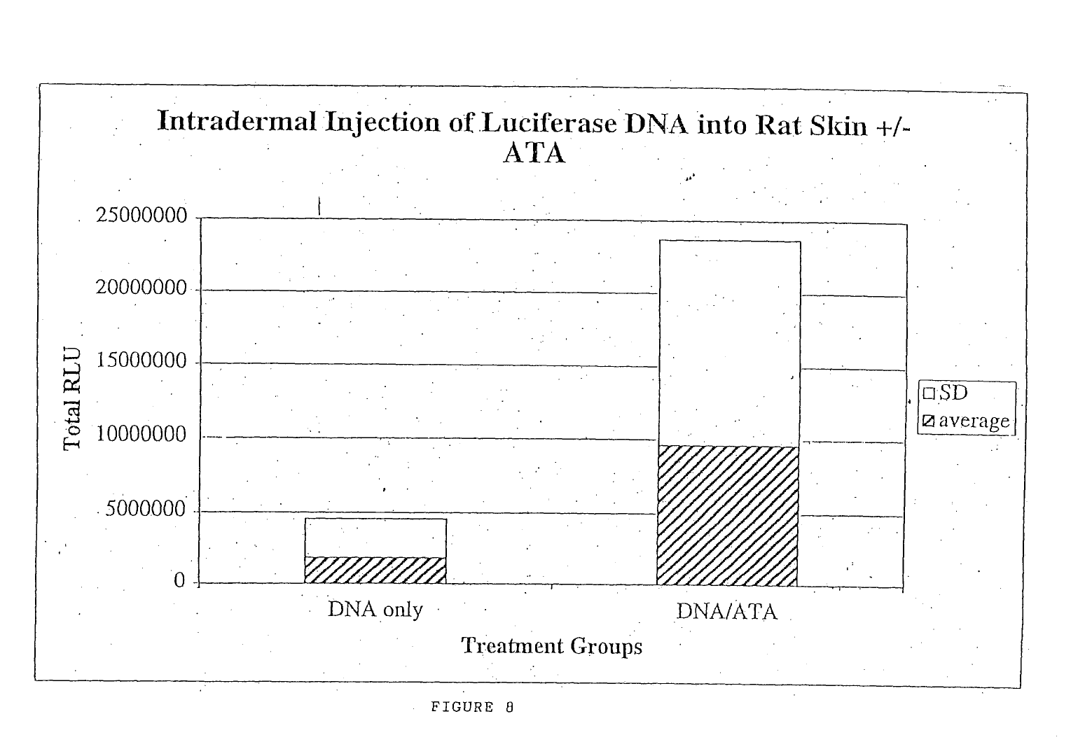

Extending the experiment from mouse to rat, the activity of free plasmid DNA +/− ATA was tested in rat skin tissues. The intradermal injection procedure was identical to that described above for mice. Eleven mice were in each group. The results are shown in FIG. 8. Statistical analysis of the results shows p=0.05.

The experiment was further extended from murine to primate, testing direct intradermal injection on macaques. The control solution was identical to that described for the mice and rat protocols. The test solution contained 100 μg of pND21ux plasmid and 50 μg of ATA in 100 μl water. Ten macaques were included in the control group and twelve were included in the test group. The results are shown below in Table 3. On average, the test group showed an 11 fold enhancement of reporter gene expression. Statistical analysis of the results shows p ranges from 0.048 (one tail) to 0.095 (two tail).

TABLE 3
Intradermal Injection of Luciferase DNA into
Macaque Skin +/− ATA

| DNA (control in RLU) | DNA + ATA (test in RLU) |
|---|---|
| 18600 | 53350 |
| 25125 | 14025 |
| 19825 | 21225 |
| 16375 | 8075 |
| 15600 | 463975 |
| 48825 | 1294200 |
| 26225 | 12750 |
| 7775 | 11825 |
| 8350 | 559000 |
| 9225 | 194575 |
| | 28900 |
| | 19125 |
| Average: 19,592.5 | Average: 223,418.75 |

### Example V

### ATA Enhancing Immune Response in Vivo

To confirm the utility of nuclease inhibitors in combination with direct DNA delivery for the purpose of eliciting immune responses to encoded antigens, the activity of free plasmid DNA encoding a test antigen (beta-galactosidase)+/− ATA was tested. Mice were innoculated with 100 micrograms of pND2beta (encoding beta-galactosidase) via intradermal, intranasal, intramuscular and intratracheal routes using the same techniques described above. ATA dose was 1.0 microgram/gram animal weight. Animals were vaccinated once with plasmid, and then serum (weeks 0, 2, 4) and lung lavage fluid (week 4) was sampled and analyzed for antibody response to the encoded test antigen by standard ELISA assay methods employing purified beta-galactosidase protein. The results are shown below in table 4. Beta-galactosidase assays showed increased IgG and IgA responses to intradermal (ID), intranasal (IN), intramuscular (IM) and intratracheal (IT) pND2beta plasmid innoculations.

TABLE 4
Beta-galactosidase ELISA assays

| | | | | Week 4 |
|---|---|---|---|---|
| Treatment | Week 0 | Week 2 | Week 4 | Lung |
| group | Serum IgG | Serum IgG | Senim IgG | Lavage IgA |
| ID + ATA | − | + | + | + |
| IN + ATA | − | + | + | + |
| IT + ATA | − | − | + | − |
| IM + ATA | − | − | − | − |
| ID − ATA | − | − | − | − |
| IN − ATA | − | − | − | − |
| IT − ATA | − | − | − | − |
| IM − ATA | − | − | − | − |

(+) indicates increased antibody response relative to pND2betas plasmid without added cofactors.

### Example VI

### DNAse Activity in Lung Fluid

Lung lavage fluid was obtained from mice, macaques and humans. The mouse experiments involved 4 test animals (A, B, C, and D) and one plasmid control. The macaque experiments involved 3 test animals (J788, J740, and J628) and one plasmid control. The human experiments involved six test patients (A, B, C, D, E, and F) and one plasmid control.

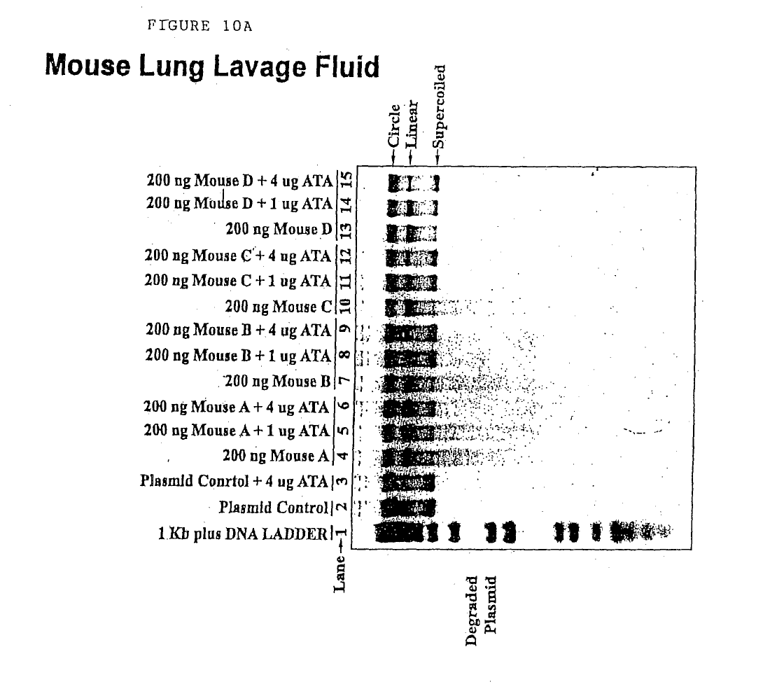

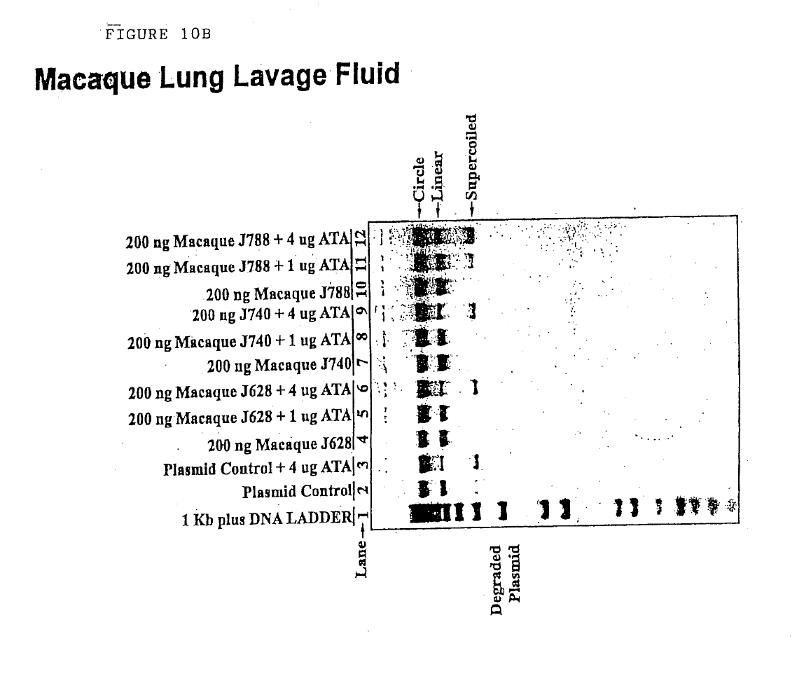

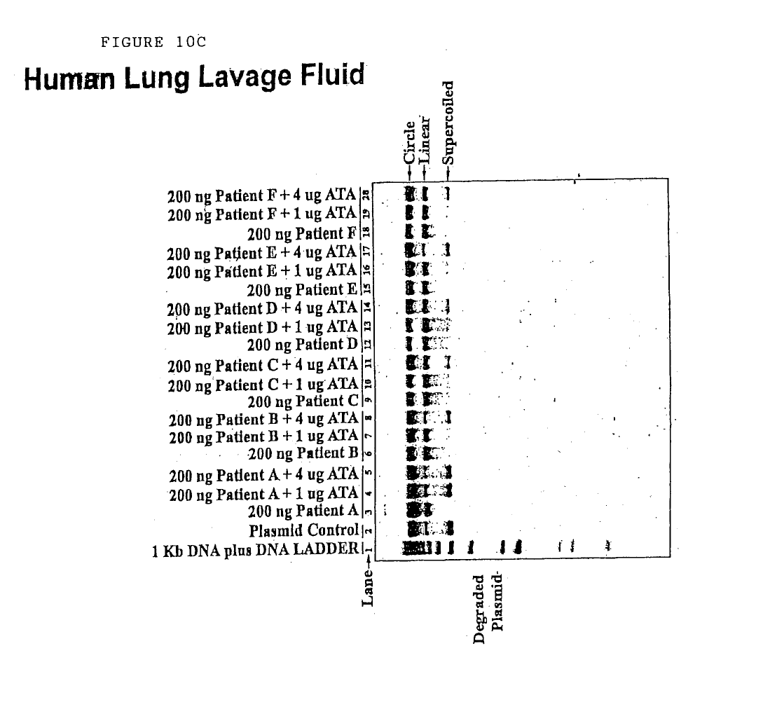

Two hundred nanograms of lung lavage protein was incubated at 37° C. with 1 μg of pND2Lux in a 1 mM solution of MnCl₂ for 2 hours. Varying amounts of ATA were added (1 or 4 μg). Mass of protein was determined by Bradford Assay and normalized for all samples. The resulting products were analyzed by ethidium bromide staining after agarose gel electrophoresis. Results are shown in FIGS. 10A (mouse), 10B (macaque), and 10C (human). In summary, a key advantage associated with this system appears to be the development of a simple, nontoxic, non-immunogenic preparation compatible with repeat administration to respiratory tissues. While the invention has been described in detail, and with reference to specific embodiments thereof, it will be apparent to one with ordinary skill in the art that various changes and modifications can be made therein without departing from the spirit and scope thereof. All references cited herein are incorporated by reference in their entirety.

What is claimed:

1. A method for enhancing the in situ expression of a polynucleotide of interest in target cells or tissues comprising administering a transfection enhancing agent in combination with a free polynucleotide preparation.

2. The method of claim 1 wherein said cells are present in a system selected from the group consisting of or the intact animal, a primary cell culture, explant culture and a transformed cell line.

3. The method of claim 1 wherein said transfection enhancing agent comprises a direct competitive nuclease inhibitor.

4. The method of claim 3 wherein said nuclease inhibitor comprises a DNAse inhibitor.

5. The method of claim 5 wherein said nuclease inhibitor is selected from the group consisting of a polyclonal nuclease antibody, actin or an actin derivative, and aurin tricarboxylic acid or a functionally derivative thereof.

6. The method of claim 1 wherein said transfection enhancing agent comprises aurin tricarboxylic acid or a functionally derivative thereof.

7. The method of claim 1 wherein said free polynucleotide preparation comprises at least one polynucleotide interest operably linked to expression elements required for expression in said target cell or tissue.

8. The method of claim 1 wherein said polynucleotide preparation is in the form of a pharmaceutically acceptable formulation.

9. The method of claim 32 wherein said formulation is selected from the group consisting of liquids, aerosols, dry powder aerosol, lipid delivery systems, and charged polymers.

10. The method of claim 1 wherein said polynucleotide of interest encodes an agent selected from the group consisting of vaccine antigens, therapeutic agents, and immunostimulating agents.

11. The method of claim 1 wherein said polynucleotide of interest is an RNA molecule.

12. The method of claim 11 wherein said RNA molecule is selected from the group consisting of ribozymes, catalytic RNA and antisense RNA.

13. The method of claim 1 wherein said cells are eukarytoic cells in vitro.

14. The method of claim 1 wherein said cells are prokaryotic cells in vitro.

15. The method of claim 1 wherein said cells or tissues are selected from the group consisting of embryos, embryonic tissues, fetal tissues, oocytes, embryonic and pleuripotent tissue or cells.

16. The method of claim 1 wherein said cells or tissues are selected from the group consisting of hepatic tissues, pancreatic tissues, mucosal tissues, respiratory tissues, skeletal muscle, cardiac muscle, vascular endothelium, liver, tumors, skin, thyroid, thymus, synovium, and brain.

17. The method of claim 1 wherein said tissues are respiratory tissues, said respiratory tissues are selected from the group consisting of oropharyngeal mucosa, nasopharyngeal mucosa, conducting airway epithelium, and pulmonary parenchyma.

18. The method of claim 1 wherein said tissues are mucosal tissues, said mucosal tissues are selected from the group consisting of Peyer's patches, Waldeyer's rings, gut-associated lymphoid tissues, bronchial associated lymphoid tissues, nasal-associated lymphoid tissues, genital-associated lymphoid tissues, and tonsils.

19. The method of claim 1 wherein said administration is selected from the group consisting of intradermal injection, intramuscular injection, intratracheal delivery, tissue electroporation and gene gun delivery.

20. The method of claim 1 wherein said host organism is selected from the group consisting of human, bovine, ovine, porcine, feline, buffalo, canine, goat, equine, donkey, deer, and primate.

21. A composition comprising a free polynucleotide preparation and an effective amount of transfection enhancing agent.

22. The composition of claim 21 wherein said transfection enhancing agent comprises a direct competitive nuclease inhibitor.

23. The composition of claim 22 wherein said nuclease inhibitor is selected from the group consisting of a polyclonal nuclease antibody, actin or an actin derivative, and aurin tricarboxylic acid or a functionally derivative thereof.

24. The composition of claim 21 wherein said transfection enhancing agent comprises aurin tricarboxylic acid or a functionally derivative thereof.

25. The composition of claim 21 wherein said polynucleotide preparation comprises at least one polynucleotide interest operably linked to expression elements required for expression in a host organism.

26. The composition of claim 21 further comprising a pharmaceutically acceptable carrier.

27. The composition of claim 21 wherein said composition is pharmaceutically formulated for direct administration, said administration selected from the group consisting of intradermal injection, intramuscular injection, tissue electroporation, gene gun delivery, and intratracheal administration.
